(19) United States
(12) Patent Application Publication
(10) Pub. No.: US 2014/0073527 A1
(43) Pub. Date: Mar. 13, 2014

(54) METHODS FOR DIAGNOSIS AND PROGNOSIS OF CANCER

(71) Applicant: THE JOHNS HOPKINS UNIVERSITY, BALTIMORE, MD (US)

(72) Inventors: Paula Jill Hurley, Perry Hall, MD (US); Edward M. Schaeffer, Sparks, MD (US)

(73) Assignee: THE JOHNS HOPKINS UNIVERSITY, BALTIMORE, MD (US)

(21) Appl. No.: 14/022,468

(22) Filed: Sep. 10, 2013

Related U.S. Application Data
(60) Provisional application No. 61/698,893, filed on Sep. 10, 2012.

(51) Int. Cl. C12Q 1/68; G01N 33/574

(52) U.S. Cl. CPC C12Q 1/6886; G01N 33/57492
USPC 506/9; 435/6.12; 436/501; 435/7.1

(57) ABSTRACT

The present invention relates to the field of cancer. More specifically, the present invention provides methods and compositions useful for assessing prostate cancer in a patient. In a specific embodiment, a method for determining a likelihood of prostate cancer recurrence in a patient following prostectomy comprises the steps of (a) obtaining a biological sample from the patient; (b) subjecting the sample to an assay for detecting SPARCL1 expression; and (c) determining that prostate cancer is likely to recur if SPARCL1 expression is decreased relative to a reference non-prostate cancer sample.

### CROSS-REFERENCE TO RELATED APPLICATIONS

This application claims the benefit of U.S. Provisional Application No. 61/698,893, filed Sep. 10, 2012; which is incorporated herein by reference in its entirety.

### STATEMENT OF GOVERNMENTAL INTEREST

This invention was made with government support under grant no. DK081019 awarded by the NIH. The government has certain rights in the invention.

### FIELD OF THE INVENTION

The present invention relates to the field of cancer. More specifically, the present invention provides methods and compositions useful for assessing prostate cancer in a patient.

### INCORPORATION-BY-REFERENCE OF MATERIAL SUBMITTED ELECTRONICALLY

This application contains a sequence listing. It has been submitted electronically via EFS-Web as an ASCII text file entitled “P12091-02_Sequence_Listing.txt.” The sequence listing is 1,057 bytes in size, and was created on Sep. 10, 2013. It is hereby incorporated by reference in its entirety.

### BACKGROUND OF THE INVENTION

Prostate cancer is the most common non-cutaneous malignancy and the second leading cause of cancer death in U.S. men. Controversy currently exists over the best treatment strategy for men with high-risk disease (clinical stage≧T2c, Gleason score 8-10 or PSA>20 ng/ml) since 56-65% of these men recur after definitive local therapy (1-5). This highlights the need for a better understanding of the biologic determinants driving disease progression for both prognostic and therapeutic development.

We and others have recently illustrated that pathways essential for prostate organogenesis are reactivated in prostate cancer (6, 7). During organogenesis, androgens induce epithelial-mesenchymal interactions in the urogenital sinus (UGS) and drive its differentiation into a prostate (8). We examined early prostate organogenesis, shortly after initial androgen exposure, when urogenital sinus epithelia (UGE) migrate and invade into the surrounding mesenchyme (UGM) and determined that the genes defining this developmental stage were similarly regulated in the transition between low and high grade prostate cancers (6). Among these genes, SPARCL1 (SPARC-like 1/Hevin/SC1), a member of the secreted protein, acidic and rich in cysteine (SPARC) family of matricellular proteins, was down regulated specifically during embryonic periods of androgen induced epithelial invasion (6) and in aggressive prostate cancers (6, 9). Sparcl1 has been shown to mitigate adhesion and to inhibit both fibroblast migration and wound healing (10). The mechanisms through which Sparcl1 regulates cellular adhesion and migration are not well understood; however, Sparcl1 has been shown to bind Type I collagen, a component of the extracellular matrix that potentiates tumor cell migration and invasion (11-13). While the C-terminal domain of SPARCL1 is highly homologous to SPARC, an inhibitor of prostate tumorigenesis and progression (14), the relationship of SPARCL1 itself to prostate cancer aggressiveness has not been well characterized.

### BRIEF DESCRIPTION OF THE FIGURES

FIG. 1: Sparcl1 inhibits androgen-induced fetal prostate bud elongation. (A) Sparcl1 expression in male mouse e17.5 UGS as detected by IHC. (B) Sparcl1 expression examined by QT-PCR during prostate development. Statistical analysis performed by 1-way ANOVA with Newman-Keuls post-hoc test (mean±SEM; n≧3; **P<0.0001). (C) Male e15.5 UGS cultured in vitro with vehicle or Sparcl1 (10 μg/ml) for 7 days (n≧13) and examined by IHC. Black box indicates bud. (D) Sparcl1 inhibits bud number, but not significantly as measured by IHC (n=4). (E) Sparcl1 significantly inhibits bud length in UGS cultured in vitro. Bud length determined from photomicrographs for vehicle (n=43) and Sparcl1 (n=30) treated UGS (n=3 UGS); *P=0.01. (F-G) Sparcl1 does not inhibit epithelial proliferation as examined by IHC for Ki67 in UGS in vitro cultures. Ki67 positive and negative cells within the epithelial bud were counted from IHC sections of e15.5 male UGS cultured in vitro with vehicle or Sparcl1 (n=3 UGS). Statistical analysis for C-G performed by Student's t test (mean±SEM).

FIG. 2: Sparcl1 expression is decreased during prostate regeneration in adult mouse. (A, B) Decreased Sparcl1 protein (A) and gene (B) expression during androgen induced re-growth determined by IF (A) and QT-PCR (B) as compared in adult mouse prostate, adult mouse prostate 3 weeks following castration and adult mouse prostrate treated with DHT for 3 days following castration (regenerating prostate) (mean±SD; n=3).

FIG. 3: Sparcl1 restricts benign prostate epithelial cell invasion. (A, B) Sparcl1 inhibits prostasphere number (A) and size (B). Adult mouse prostate epithelial cells disassociated into single cells, cultured in Matrigel and treated with Sparcl1 (10 μg/ml) or vehicle for 14 days to form prostaspheres. Statistical analysis performed by Student's t test (mean±SEM; n=4; *P≦0.005).

FIG. 4: SPARCL1 inhibits adhesion, migration, and invasion of prostate cancer cells. (A, B) Adhesion of PC3 cells following incubation on a Type I collagen matrix containing BSA (10 μg/ml) or SPARCL1 (10 μg/ml) (n=3). Arrows indicate adhered cells. (C) Migration of PC3 cells incubated with BSA (10 μg/ml) or SPARCL1 (10 μg/ml) across a filter for 20 hrs (n=3). (D) Cell adhesion and migration recorded by time-lapse microscopy for 22 hrs of PC3 cells on a Type I collagen matrix containing SPARCL1 (10 μg/ml) or BSA (10 μg/ml). (E) Invasion of PC3 cells incubated with BSA (10 μg/ml) or SPARCL1 (10 μg/ml) of Type I collagen or Matrigel coated filters for 20 hrs (n=3). Statistical analysis performed by Student's t test (mean±SEM; *P≦0.005).

FIG. 5: SPARCL1 inhibits Type I collagen induced RHOC mediated migration. (A) PC3 cells grown on a Type I collagen matrix containing SPARCL1 (10 μg/ml) or BSA (10 μg/ml). Specific IP of activated (GTP bound) RHOA/B/C and IB for RHOA and RHOC. Pre-IP lysates were examined for total RHOC, RHOA, GAPDH, and SPARCL1 expression. ImageJ quantification of activated RHOC (normalized to total pre-IP RHOC). Statistical analysis performed by Student's t test (mean±SEM; n=4; *P=0.02). (B) PC3 cells transiently transfected with RHOC or constitutively active RHOC (G14V), treated with SPARCL1 (10 μg/ml) or vehicle and allowed to migrate across a filter for 20 hrs. Statistical analysis performed by Student's t test (mean 19±SEM; n=3; *P=0.013). (C) PC3 cells transiently transfected with pcDNA3.1- or hSPARCL1/pcDNA3.1, treated with isotype control or a α2β1-integrin blocking antibody and then grown on a Type I collagen matrix. Specific IP of activated (GTP bound) RHOA/B/C and IB for RHOC. Pre-IP lysates IB for total RHOC and GAPDH expression.

FIG. 6: Loss of SPARCL1 expression correlates with Gleason grade and is an independent marker for prostate cancer recurrence. (A, B) SPARCL1 expression is inversely proportional to prostate cancer Gleason grade as determined by IHC in prostate adenocarcinoma Gleason sum 5 (n=4), 6 (n=16), 8 (n=10), and 9 (n=8), and benign adjacent glands (n=20) from radical prostatectomies as JHU Gleason grade TMAs. Statistical analysis performed by 1-way ANOVA with Bonferroni post-hoc test (mean±SEM; *P≦0.002). (C) SPARCL1 expression is inversely proportional to prostate cancer Gleason grade. Analysis performed on data sets from Taylor et al. for SPARCL1 gene expression (27). Statistical analysis performed by 1-way ANOVA. *PCA vs. benign adjacent and **Met vs. PCA P≦0.01. (D, E) Loss of SPARCL1 expression is prognostic of prostate cancer recurrence. (D) Kaplan-Meier curves for SPARCL1 in a high-risk prostate cancer cohort from the Mayo Clinic for BCR (P=0.007), MET (P=0.0009), and PCSM (P=0.07) endpoints (n=235). (E) Kaplan-Meier curves for SPARCL1 in a Gleason sum 7 cohort (n=119, P=0.046) and a Gleason sum≧8 cohort (n=98, P=0.011) from the Mayo Clinic for MET free survival.

FIG. 7: Sparcl1 protein expression is increased in prostate epithelial cells following castration. (A) Sparcl1 expression in the adult mouse prostate as determined by IHC. (B) Sparcl1 expression occurs in a sub-population of basal cells as determined by IF co-localization with CK14 in adult mouse prostate. (C) Increased Sparcl1 protein expression following castration (three weeks) occurs predominantly in luminal prostate epithelial cells as determined by IF co-localization with CK8 and CK14. (D) SPARCL1 protein expression in adult human benign prostate as determined by IF co-localization with p63 and CK8.

FIG. 8: SPARCL1 does not affect cellular proliferation or death in human prostate cancer cell lines. (A) SPARCL1 does not affect cell growth in human prostate cancer cell lines. PC3, DU145, and CWR22RV1 cells were treated with vehicle or recombinant hSPARCL1 (10 μg/ml) for 0, 2, 3, and 4 days. Cell growth was analyzed by Thiazolyl Blue Tetrazolium Bromide (MTT) assay (mean±SD; n=3). (B) SPARCL1 does not affect DNA synthesis in prostate cancer cells. PC3 cells were treated with vehicle or recombinant hSPARCL1 (10 μg/ml) for 24 hours, incubated with 5-ethynyl-2′-deoxyuridine (EdU), and then examined by flow cytometry for EdU incorporation as a measure of DNA synthesis and S-phase progression. (C) SPARCL1 does not affect cell cycle progression in prostate cancer cells. Asynchronous PC3 cells were treated with vehicle or recombinant hSPARCL1 (10 μg/ml) for 24 hours. All cultures were then treated with the microtubule inhibitor nocodazole to block cells in mitosis and thereby preventing nascent cells from re-entering the 2N population. Cell cycle distribution was measured by flow cytometry. (D) SPARCL1 does not cause apoptosis in prostate cancer cells. PC3 cells were treated with vehicle, recombinant hSPARCL1 (10 μg/ml), or Bleomycin (200 μg/ml) for 48 hours. Cell death was examined by flow cytometry following annexin staining (mean±SD; n=3).

FIG. 9: Sparcl1 inhibits prostasphere formation following initiation. (A) Sparcl1 does not inhibit prostasphere proliferation or differentiation as analyzed by IF for Ki67 (proliferation) and CK8, CK14, and p63 (differentiation) in vehicle and recombinant mSparcl1 (10 μg/ml) treated prostaspheres. Despite overall differences in size between vehicle and Sparcl1 treated prostaspheres, sections of approximate equal diameter were chosen for comparable analysis of differentiation and proliferation markers. (B,C) Adult mouse prostate epithelial cells established in Matrigel for 0, 2, or 4 days prior to treatment with recombinant mSparcl1 (10 μg/ml) or vehicle (mean±SD; *P≦0.003). (D) SPARCL1 restricts benign prostate epithelial prostasphere formation. Adult human primary benign prostate epithelial cells from deceased organ donors (PrEC), cultured in Matrigel and treated with recombinant hSPARCL1 (10 μg/ml) or vehicle for 14 days to form prostaspheres (mean±SEM; n=4; *P≦0.001). Statistical analysis performed by Student's t test.

FIG. 10: SPARCL1 delays/abrogates prostate cancer cell adhesion to Type I collagen. DU145, CWR22RV1, LNCaP, and PrEC cells were allowed to attach to a Type I collagen/BSA (10 μg/ml) or a Type I collagen/recombinant hSPARCL1 (10 μg/ml) matrix. Following incubation, plates were photographed and monitored for cellular adhesion. Abrogation of adhesion in LNCaP cells may be related to their relatively non-adherent nature compared to the other cell lines and cells. Statistical analysis performed by Student's t test (mean±SEM; n≧3; *P≦0.004).

FIG. 11: Sparcl1 expression is decreased in animal models of prostate adenocarcinoma. (A) Sparcl1 expression is decreased in locally invasive primary prostate adenocarcinoma as examined by IHC in benign prostate adjacent to cancer, prostate hyperplasia, and primary prostate adenocarcinoma in Hi-Myc mice (n=4). (B) Decreased Sparcl1 expression in primary prostate adenocarcinoma and metastatic lesions as determined by IHC in benign prostate adjacent to cancer, primary prostate adenocarcinoma, and metastatic lesions to the liver from TRAMP mice (n=5). (C) Sparcl1 expression is decreased in primary prostate adenocarcinoma compared to benign adjacent glands and mPIN as examined by IHC in benign prostate adjacent to cancer, PIN, and primary prostate adenocarcinoma from TRAMP mice (n=5).

FIG. 12: SPARCL1 gene expression inversely correlates with prostate cancer aggressiveness. (A) SPARCL1 gene expression inversely correlates with prostate cancer Gleason grade. Analysis performed on data sets from Ross et al. for SPARCL1 gene expression. *Benign prostatic epithelial vs. PCA P≦0.005. (B) SPARCL1 gene expression is inversely proportional to prostate cancer aggressiveness. One way Anova analysis was performed on data sets from Chandran et al. for SPARCL1 gene expression. *Donor normal vs. prostate carcinoma and **prostate carcinoma vs. metastatic prostate cancer P≦0.00005. (C) Data obtained from Oncomine™ (Compendia Bioscience, Ann Arbor, Mich.) was used for analysis and visualization. In (1) Ramaswamy et al., primary prostate tumor (n=10) vs. metastasis (n=4) P=0.008. In (2) Ramaswamy 2 et al., primary prostate tumor (n=10) vs. metastasis (n=3) P=0.028. In (3) Varambally et al., primary prostate tumor (n=7) vs. metastasis (n=6) P=0.026. In (4) Lapointe et al., primary prostate cancer (n=62) vs. metastasis (n=8) P=5.79E-5. In (5) LaTulippe et al., primary prostate cancer (n=23) vs. metastasis (n=9) P=2.75E-5. In (6) Holzbeierlein et al., primary prostate cancer (n=40) vs. metastasis (n=9) P=4.41E-6. In (7) Yu et al., primary prostate cancer (n=64) vs. metastasis (n=24) P=1.71E-10. (D) Data obtained from Oncomine™ (Compendia Bioscience, Ann Arbor, Mich.) was used for analysis and visualization. In Tomlins et al., SPARCL1 gene expression is not altered significantly in BPH (P=0.771) or PIN (P=0.054) compared to benign epithelia adjacent to prostate cancer. (E) SPARCL1 expression as measured by IHC is not decreased significantly in hPIN (n=12) as compared to benign epithelia adjacent to prostate cancer (n=22) P=0.652. (F, G) RHOC gene expression does not correlate with prostate cancer Gleason grade. In silico analysis was performed on data sets from Taylor et al. and Ross et al. for RHOC gene expression. In Taylor et al. (F), RHOC in benign prostate adjacent to cancer vs. PCA G3+3 P=0.032, benign prostate adjacent to cancer vs. G3+4 P=0.013, benign prostate adjacent to cancer vs. PCA G4+3 P=0.208, benign prostate adjacent to cancer vs. PCA G4+4 P=0.018, benign prostate adjacent to cancer vs. PCA G4+5 P=0.034, and benign prostate adjacent to cancer vs. MET P=0.657. In Ross et al. (G), RHOC in benign prostatic epithelial vs. PCA epithelia G3+3=6 P=0.905 and benign prostatic epithelial vs. PCA epithelia G4+4=8 P=0.106. (H) SPARCL1 gene expression is reduced in human prostate cancer cell lines compared to primary benign human prostate epithelial cells. Expression of SPARCL1 mRNA was determined by QT-PCR in human prostate cell lines (LNCaP, 22RV1, and PC3) and in human primary prostate epithelial cells from deceased organ donors (PrEC). (I) SPARCL1 gene expression inversely correlates with cancer aggressiveness in multiple solid malignancies. Data obtained from Oncomine™ (Compendia Bioscience, Ann Arbor, Mich.) was used for analysis and visualization. In Sanchez-Carbayo et al., normal bladder (n=48) vs. infiltrating urothelial carcinoma (n=81) P=5.09E-29. In Richardson et al., normal breast (n=7) vs. ductal breast carcinoma (n=40) P=4.07E-14. In Notterman et al., normal colon (n=18) vs. colon adenocarcinoma (n=18) P=3.44E-8. In Sabates Bellver et al., normal rectum (n=32) vs. rectal adenoma (n=7) P=4.70E-5. In Estilo et al., normal tongue (n=26) vs. tongue squamous cell carcinoma (n=32) P=1.50E-6. In Bhattacharjee et al., normal lung (n=17) vs. lung adenocarcinoma (n=139) P=5.33E-7. In Talantov et al., normal skin (n=7) vs. cutaneous melanoma (n=45) P=4.40E-6. From unpublished Cancer Genome Atlas normal ovary vs. ovarian serous cystadenocarcinoma P=1.69E-10. (J) SPARCL1 gene expression is down-regulated in metastases compared to the primary tumor in multiple solid malignancies. Data obtained from Oncomine™ (Compendia Bioscience, Ann Arbor, Mich.) was used for analysis and visualization. In Segal et al., sarcoma 1 (n=29) vs. metastasis (n=4) P=0.009. In Segal et al., sarcoma 2 (n=46) vs. metastasis (n=4) P=0.007. In Liao et al., liver carcinoma (n=4) vs. metastasis (n=6) P=0.007. In Xu et al., melanoma (n=31) vs. metastasis (n=52) P=8.83E-5. In Radvanyi et al., breast carcinoma (n=47) vs. metastasis (n=7) P=2.4E-4. In Ki et al., colorectal cancer (n=52) vs. metastasis (n=28) P=3.28E-4. In Bhattachargee et al., lung cancer (n=123) vs. metastasis (n=7) P=0.004.

FIG. 13: Multivariable analysis of The Johns Hopkins University cohort demonstrates that loss of SPARCL1 expression is independently associated with prostate cancer recurrence. (A) Characteristics of The Johns Hopkins University Progression Cohort recurrence cases and matched controls. Recurrence cases as measured by biochemical recurrence (PSA≧0.2 ng/ml), metastasis, or prostate cancer death after surgery were matched by Gleason sum, pathologic stage, age, and race/ethnicity to non-recurrent controls. Conditional logistic regression was used to estimate the odds ratio of recurrence taking in account the matching factors and adjusting for year of surgery, pre-surgical PSA level, positive surgical margins, and residual difference in pathologic stage and grade (n=136). (B) Variation of SPARCL1 expression within Gleason grade. SPARCL1 expression varies within Gleason grade as measured by IHC. (C) SPARCL1 protein expression was determined by IHC and quantified using TMAJ software on a series of TMAs containing prostate adenocarcinomas matched for Gleason grade, pathologic stage, age, and race/ethnicity but differing in recurrence as measured by biochemical recurrence (PSA≧0.2 ng/ml), metastasis, or prostate cancer death after surgery from The Johns Hopkins University progression cohort (n=136). Multiple parameters of SPARCL1 expression criteria in multivariate analysis show the loss of SPARCL1 expression in prostate adenocarcinomas is significantly associated with prostate cancer recurrence independent of Gleason grade, pathologic stage, age, race/ethnicity, PSA, and other clinical variables.

FIG. 14: Multivariable analyses of the Mayo Clinic cohort demonstrate that SPARCL1 expression is significantly prognostic of BCR, MET, and PCSM. (A) Characteristics of the Mayo Clinic High Risk Progression Cohort. The percentage of biochemical recurrence (BCR) as defined by two consecutive increases of ≧0.2 ng/ml PSA after radical retro-pubic prostatectomy, metastatic progression (MET) as defined by a positive CT or bone scan, prostate cancer specific mortality (PCSM), Gleason sum (GS), and positive surgical margins (SMS) events in the dataset (n=235). (B) Multivariable Cox regression survival analyses for SPARCL1 gene expression in the Mayo Clinic high-risk prostate cancer progression cohort for BCR, MET, and PCSM endpoints (n=235). Gleason sum (GS), seminal vesicle invasion (SVI) (pathologic stage), extracapsular extension (ECE) (pathologic stage), and positive surgical margins (SMS). (C) Multivariable Cox regression survival analyses of the Mayo Clinic cohort stratified by Gleason sum (7, n=119 and ≧8, n=98) demonstrate that SPARCL1 gene expression is significantly prognostic of MET.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The present invention is based, at least in part, on the discovery that downregulation of SPARCL1 expression is prognostic of prostate cancer recurrence after surgery. As described herein, the present inventors describe specific roles for SPARCL1 that originate during prostate formation and are reprised in prostate cancer progression. The present inventors demonstrate that SPARCL1 restricts epithelial invasion both during androgen induced prostate development and in prostate cancer. Mechanistically, SPARCL1 is shown to block the activation of the Ras homolog gene family, member C(RHOC), thereby inhibiting cellular movement. It is consistently found that SPARCL1 is not only down-regulated in localized, high grade prostate cancer lesions, but is also further repressed in prostate cancer metastases, thus implicating SPARCL1 as a biomarker of lesions with metastatic potential. Consistent with this, in multivariate analyses, the present inventors found that the loss of SPARCL1 expression is significantly prognostic of metastatic recurrence after surgery. These findings suggest that loss of SPARCL1 leads to an increase in the migratory potential of prostatic epithelial cells, resulting in a more aggressive and invasive phenotype and thereby driving disease recurrence. These data support the potential utility of SPARCL1 as an independent prognostic factor for prostate cancer progression.

Accordingly, in one aspect, the present invention provides methods for determining a likelihood of prostate cancer recurrence in a patient following prostectomy. In a specific embodiment, the method comprises the steps of (a) obtaining a biological sample from the patient; (b) subjecting the sample to an assay for detecting SPARCL1 expression; and (c) determining that prostate cancer is likely to recur if SPARCL1 expression is decreased relative to a reference non-prostate cancer sample. In another embodiment, a method for determining a likelihood of prostate cancer recurrence in a patient following prostectomy comprises the steps of (a) obtaining a prostate tissue sample from the patient; (b) performing an assay on the sample to measure SPARCL1 expression; (c) providing a reference non-prostate cancer tissue sample; (d) comparing the level of SPARCL1 expression from the prostate tissue sample of the patient to the level of SPARCL1 expression in the reference non-prostate cancer tissue sample; and (e) determining that prostate cancer is likely to recur when the level of SPARCL1 expression in the prostate tissue sample of the patient is decreased relative to the level of SPARCL1 expression in the reference non-prostate cancer tissue sample.

In another aspect, the present invention provides methods for predicting metastasis in prostate cancer patient. In a specific embodiment, the method comprises the steps of (a) obtaining a biological sample from the patient; (b) subjecting the sample to an assay for detecting SPARCL1 expression; and (c) determining that metastasis is likely to occur if SPARCL1 expression is decreased relative to a reference non-metastatic prostate cancer sample. In another embodiment, a method for predicting metastasis in prostate cancer patient comprises the steps of (a) obtaining a prostate tissue sample from the patient; (b) performing an assay on the sample to measure SPARCL1 expression; (c) providing a reference non-prostate cancer tissue sample; (d) comparing the level of SPARCL1 expression from the prostate tissue sample of the patient to the level of SPARCL1 expression in the reference non-prostate cancer tissue sample; and (e) determining that metastasis is likely to occur when the level of SPARCL1 expression in the prostate tissue sample of the patient is decreased relative to the level of SPARCL1 expression in the reference non-prostate cancer tissue sample.

In yet another aspect, the present invention provides methods for identifying prostate cancer lesions with metastatic potential in a patient. In a particular embodiment, the method comprises the steps of (a) obtaining a biological sample from the patient; (b) subjecting the sample to an assay for detecting SPARCL1 expression; and (c) determining that the prostate cancer lesions have metastatic potential if SPARCL1 expression is decreased relative to a reference non-metastatic prostate cancer sample. In another embodiment, a method for identifying prostate cancer lesions with metastatic potential in a patient comprises the steps of (a) obtaining a prostate tissue sample from the patient; (b) performing an assay on the sample to measure SPARCL1 expression; (c) providing a reference non-prostate cancer tissue sample; (d) comparing the level of SPARCL1 expression from the prostate tissue sample of the patient to the level of SPARCL1 expression in the reference non-prostate cancer tissue sample; and (e) determining that the prostate cancer lesions have metastatic potential when the level of SPARCL1 expression in the prostate tissue sample of the patient is decreased relative to the level of SPARCL1 expression in the reference non-prostate cancer tissue sample.

The present invention further provides methods for diagnosing prostate cancer or a likelihood thereof in a patient. In a specific embodiment, the method comprises the steps of (a) obtaining a biological sample from the patient; (b) subjecting the sample to an assay for detecting SPARCL1 expression; and (c) determining that the cancer lesions have metastatic potential if SPARCL1 expression is decreased relative to a reference non-prostate cancer sample. In another embodiment, a method for identifying a patient as having prostate cancer comprises the steps of (a) obtaining a prostate tissue sample from the patient; (b) performing an assay on the sample to measure SPARCL1 expression; (c) providing a reference non-prostate cancer tissue sample; (d) comparing the level of SPARCL1 expression from the prostate tissue sample of the patient to the level of SPARCL1 expression in the reference non-prostate cancer tissue sample; and (e) identifying the patient as having prostate cancer when the level of SPARCL1 expression in the prostate tissue sample of the patient is decreased relative to the level of SPARCL1 expression in the reference non-prostate cancer tissue sample.

In certain embodiments, the reference non-prostate cancer tissue sample is a sample from benign prostate tissue. In a specific embodiment, the benign prostate tissue is from the patient. In fact, in particular embodiments, the sample is from adjacent benign prostate tissue. The assay used to measure SPARCL1 expression can be a PCR assay. In another embodiment, the assay is an immunohistochemical assay. In another embodiments, the assay utilizes mass spectrometry.

Enrichment of embryonic gene expression signatures has been demonstrated in multiple solid malignancies, substantiating the paradigm of embryonic reawakening in cancer and the utility of embryonic systems to model cancer progression (6, 7, 49). With this approach, we show that the developmental regulation of Sparcl1 expression is paralleled in prostate cancer. Similar to periods of physiologic growth, we illustrate an inverse correlation between SPARCL1 expression and high grade localized prostate cancer as well as metastatic lesions. Consistent with its role in physiologic epithelial invasion during development, we demonstrate that the loss of SPARCL1 expression increases the migratory and invasive properties of prostate epithelial cells through a RHOC mediated process. We further demonstrate that loss of SPARCL1 expression is not only associated with aggressive disease, but is also independently associated with disease recurrence following treatment, indicating that loss of SPARCL1 expression in the primary tumor may drive metastasis rather than solely being a marker of metastatic lesions. Together, these data suggest that by suppressing RHOC mediated migration, SPARCL1 plays a key role in modulating the metastatic potential of cancer and further defines loss of SPARCL1 as an early marker of aggressive prostate cancer.

Recent studies show Type I collagen stimulation of the α2β1-integrin promotes prostate cancer cell migration through RHOC activation (12). We demonstrate here that SPARCL1, a Type I collagen binding protein, attenuates Type I collagen induced RHOC activation and this corresponds to decreased RHOC mediated migration in the prostate (11). RHOC has been shown to affect the localization of active Rac 1, a distinct member of the RHO family (23). Consistent with that study and our finding that SPARCL1 negatively regulates RHOC activity, a separate report using a small molecule inhibitor against Rac1 suggests that Sparcl1 inhibits Rac1-dependent migration in fibroblasts (10). Further, although RHOC expression is elevated in multiple cancers including breast (51), bladder (52), and non-small cell lung carcinoma (53), its expression levels do not correlate with prostate cancer aggressiveness. This suggests that unlike other tumors which over express RHOC, prostate cancers may regulate RHOC mediated migration via modulation of SPARCL1 expression. Together, these studies suggest a role for SPARCL1 as a master regulator of RHOC-RAC1 mediated cellular migration and invasion.

We demonstrate that SPARCL1 may have clinical utility as a prognostic marker that is independently associated with prostate cancer recurrence. Thus SPARCL1 expression may identify patients who are in greatest need of additional therapies. In addition, we outline a key biologic role for SPARCL1 in prostate cancer. Thus it is possible that treatments targeting this pathway could attenuate the metastatic potential of localized cancers and we believe that further understanding of the factors modulating SPARCL1 will have important clinical implications for both prognostic and therapeutic development.

In another aspect, SPARCL1 expression is prognostic of recurrence of other cancers including, but not limited to, bladder, breast, colorectal, skin, tongue and ovarian. Thus, the present invention provides methods for predicting metastasis in a cancer patient. In a specific embodiment, the method comprises the steps of (a) obtaining a biological sample from the patient; (b) subjecting the sample to an assay for detecting SPARCL1 expression; and (c) determining that metastasis is likely to occur if SPARCL1 expression is decreased relative to a reference non-metastatic cancer sample.

The present invention also provides methods for identifying prostate cancer lesions with metastatic potential in a patient. In a particular embodiment, the method comprises the steps of (a) obtaining a biological sample from the patient; (b) subjecting the sample to an assay for detecting SPARCL1 expression; and (c) determining that the cancer lesions have metastatic potential if SPARCL1 expression is decreased relative to a reference non-metastatic cancer sample.

In another aspect, the present invention provides methods for diagnosing cancer or a likelihood thereof in a patient. In a specific embodiment, the method comprises the steps of (a) obtaining a biological sample from the patient; (b) subjecting the sample to an assay for detecting SPARCL1 expression; and (c) determining that the cancer lesions have metastatic potential if SPARCL1 expression is decreased relative to a reference non-cancer sample.

In such embodiments, the cancer is any cancer in which SPARCL1 expression is decreased relative to a non-cancer reference. More specifically, the cancer includes, but is not limited to, bladder, breast, colorectal, skin, tongue and ovarian.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

RNA Isolation and Real-Time Reverse Transcription/Polymerase Chain Reaction Assays.

Total RNA was purified using the RNeasy Mini-kit (Qiagen). First strand cDNA was synthesized using random hexamer primers (Applied Biosystems) and Ready-To-Go You-Prime First-Strand Beads (GE Healthcare) according to manufacturer's instructions. Quantitative PCR was performed using iQ SYBR Green Supermix (BioRad) with primers specific to human SPARCL1 set one F/R: (5′GTTCCTTCACAGATTCTAACCA3′) (SEQ ID NO:1) (5′TTTACTGCTCCTGTTCAACTG3′) (SEQ ID NO:2) set two F/R: (5′ATCATTCCAAACCAACTGCT3′) (SEQ ID NO:3) (5′GACTGTTCATGGCTTTCCTC3′) (SEQ ID NO:4). Bio-Rad MyiQ software was used to calculate threshold cycle values for SPARCL1 and the reference gene hypoxanthine phosphoribosyltransferase (HPRT). Quantitative PCR was performed using TaqMan Universal PCR Master Mix (Applied Biosystems) with TaqMan primers specific to mouse Sparcl1 (Applied Biosystems). Applied Biosystems software was used to calculate threshold cycle values for Sparcl1 and the reference gene hypoxanthine phosphoribosyltransferase (HPRT).

In Vitro Organ Culture.

The protocol was approved by the Johns Hopkins University Animal Care and Use Committee. The UGS was harvested from e15.5 males and then incubated in UGS media: DMEM-F12 (Invitrogen), Nonessential amino acids (Cellgro), ITS media (Sigma), Pen/Strep (Invitrogen), 1 g/L D-glucose (Sigma), and L-Glutamine (Invitrogen) with recombinant murine Sparcl1 (mSparcl1) (10 μg/ml) (R&D 4547-SL) or vehicle for 2 hours at 4° C. The UGS was placed (ventral side up) on a 0.4 μm Millicell filter (Millipore) in a 6 well plate with UGS media supplemented with 10⁻⁸ M DHT and vehicle or recombinant mSparcl1 (10 μg/ml). Media was changed every 24 hours.

Immunohistochemistry, Immunofluorescence, and Immunoblotting.

For immunohistochemistry and immunofluorescence, tissues were fixed in 10% neutral buffered formalin (NBF), embedded in paraffin, sectioned, deparaffinized, steamed in Target Retrieval Solution Ready to Use (Dako) for 40 minutes, blocked with Protein Block Serum-Free (Dako), incubated with antibodies directed against CK8 (Covance, MMS-162P, 1:500-1000), CK14 (Covance, PRB155P, 1:300), pancytokeratin (Sigma, C2562, 1:400), p63 (Millipore, MAB4135, 1:100), SPARCL1 for mouse and human (Abcam, Ab-107533, 1:500), Ki67 (Abcam, Ab-15580, 1:100) in Antibody Diluent (Invitrogen). Antibodies to SPARCL1 were comprehensively tested in accordance with The Johns Hopkins Brady Urological Research Institute Prostate Specimen Repository protocols Immunohistochemistry was detected with 3,3′-Diaminobenzidine (DAB) kit (Vector Laboratories). For immunofluorescence primary antibodies were followed by Alexa Fluor Dye secondary antibodies (Invitrogen) and mounted with Vectashield hard set mounting medium with 4′-6′-diamindino-2-phenylindole (DAPI) counterstain (Vector Laboratories). Images were captured at room temperature on a Nikon E800 fluorescence microscope with 40× Plan Apo objective and a Nickon DS-QiMc camera with Nikon Elements imaging software (Version AR 3.0).

For immunoblotting, lysates were fractionated on NuPAGE gels (Invitrogen). Proteins were transferred to polyvinylidene difluoride membranes, blocked, and then incubated with antibodies directed against SPARCL1 (Abcam, Ab-107533, 1:1000), GAPDH (Santa Cruz, Sc-32233, 1:5000), RhoA (Santa Cruz, Sc-418, 1:1000), and RhoC (Cell Signaling Technology, 3430, 1:1000) according to manufacturer's recommendation. Recombinant (and endogenous) SPARCL1 yield multiple bands detected by immunoblot most likely due to post-translational modifications. Brekken et al., 52 J. HISTOCHEM. CYTOCHEM. 735-48 (2004). Blots were developed using enhanced chemiluminescence (Thermo Fisher Scientific) or Odyssey IRDye (LI-COR Biosciences).

Prostate Regeneration.

The protocol was approved by the Johns Hopkins University Animal Care and Use Committee. C57B16/J mice obtained from The Jackson Laboratory were castrated, rested for 14 days and treated with daily subcutaneous vehicle (80% glycerol trioleate in ethanol) alone or with DHT (50 mg/kg). Prostates were collected from euthanized animals and processed for histology or for RNA purification (Qiagen).

Three Dimensional Prostate Invasion Assay.

The protocol was approved by the Johns Hopkins University Animal Care and Use Committee. Prostates were harvested from adult euthanized C56BL6 mice obtained from The Jackson Laboratory, minced with a razor blade, and dissociated with 0.5% Collagenase Type II (Sigma) in DMEM (Invitrogen) with 10% FCS (Gemini Bio-Products) for 60 minutes at 37° C. with shaking. Cell clumps were pipetted every 15 minutes during digestion. Cells were centrifuged and resuspended in 0.25% trypsin for 10 minutes at 37° C., washed with PBS, re-suspended in DMEM with 10% FCS, plated on a 6 cm plate, and incubated for 2 hours at 37° C. with 5% CO₂. Non-adherent cells were passed through a 40 μm nylon mesh, washed with PBS, re-suspended in PrEBM (Lonza), and counted. 20,000 cells in PrEBM plus recombinant mSparcl1 (10 μg/ml) (R&D) or vehicle were mixed with an equal amount of Matrigel (BD Biosciences), plated around the rim of a well of a 12 well plate, allowed to solidify for 30 minutes at 37° C. with 5% CO₂, and then treated with recombinant mSparcl1 (10 μg/ml) or vehicle in PrEBM. Media was changed every 24 hours. Prostaspheres were isolated from the Matrigel with Dispase (BD Biosciences) for 30 minutes at 37° C., washed with PBS, fixed in 10% NBF, washed with PBS, embedded in 2% agarose, and processed for histology. 500 PrEC cells were used in the above protocol for PrEC prostasphere formation.

Cell Growth, Cell Cycle, and Apoptosis Assays.

Cell growth in PC3, DU145, and CWR22RV1 was assayed by incubating cells in Thiazolyl Blue Tetrazolium Bromide (MTT) for 5 minutes at room temperature (RT) with shaking and then at 37° C. for 1 hour. Cells were then incubated in DMSO for 5 minutes at RT with shaking and optical density was read at 570 nm and 690 nm.

To assay the cell cycle, PC3 were treated with recombinant human SPARCL1 (hSPARCL1) (10 μg/ml) (R&D 2728-SL) and nocodazole (to induce G2/M arrest) for 0, 6 and 18 hours. Cells were collected by incubation in trypsin/ethylenediamine-tetraacetic acid, pelleted by centrifugation and fixed in phosphate-buffered saline (PBS) containing 3.7% formaldehyde, 0.5% Nonidet P-40 and 10 μg/ml Hoechst 33258. A total of 10,000 cells were analyzed per sample on a flow cytometer (LSR11, Applied Biosystems).

Proliferation in PC3 cells was assayed using the Click-iT EdU Cell Proliferation Assay according to the manufacturer recommendation (Invitrogen). A total of 10,000 cells were analyzed per sample on a flow cytometer (LSR11, Applied Biosystems).

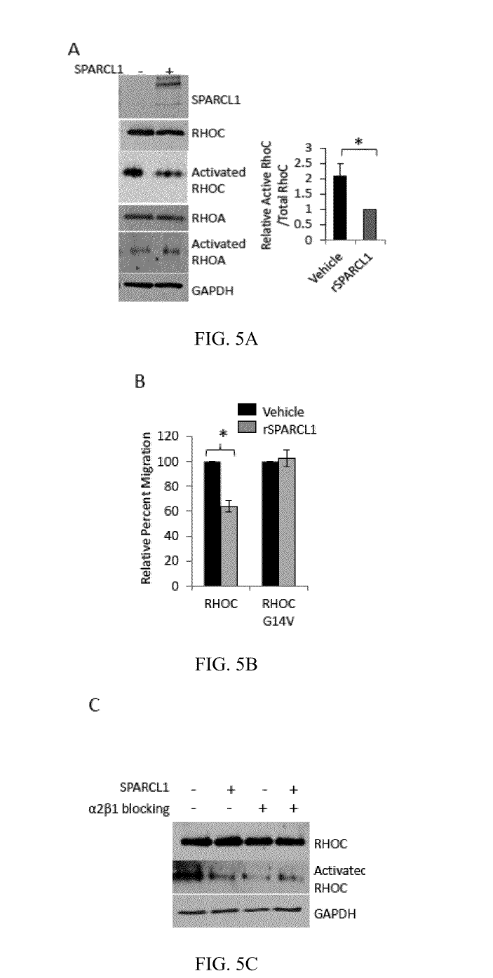

Cell death in PC3 cells was analyzed according to the manufacturer recommendation of the Vybrant Apoptosis Assay Kit with FITC Annexin (Invitrogen). A total of 10,000 cells were analyzed per sample on a flow cytometer (FACSCaliber, BD Biosciences). Cell Culture. We used multiple human prostate cancer cell lines lacking SPARCL1 expression (FIG. 5A, 12H). Human prostate cancer cell lines PC3 (gifted from John Isaacs, Johns Hopkins University, Baltimore, Md.), CWR22RV1 (gifted from John Isaacs, Johns Hopkins University, Baltimore, Md.), DU145 (gifted from John Isaacs, Johns Hopkins University, Baltimore, Md.) and LNCaP (American Type Culture Collection [ATCC]) cells were cultured in growth media (RPMI-1640 supplemented with 10% FBS). Human primary prostate epithelial cells PrEC (Lonza) were cultured in growth media (PrEBM, Lonza).

Adhesion, Migration, and Invasion Assays.

Cell adhesion was assayed in PC3, CWR22RV1, DU145, LNCaP and PrEC cells. An equal number of cells were seeded on Type I collagen/vehicle or Type I collagen/SPARCL1 coated plates. Cell adhesion was monitored and photographed using a light microscope. Cell migration was assayed using the Cell Migration Colorimetric Assay Kit (Millipore) according to the manufacturer's instructions. Cell invasion was assayed using the QCM ECMatrix Colorimetric Cell Invasion Assay (Millipore) and the QCM Collagen Colormetric Cell Invasion Assay (Millipore) according to the manufacturer's instructions. PC3 cells were seeded on Type I collagen/vehicle or Type I collagen/SPARCL1 dually coated plates and photographed every 5 minutes for 22 hours using Incucyte (Essen Bioscience) and analyzed for proliferation, adhesion and migration with Incucyte software. PC3 cells were transfected with RhoC (Missouri S&T Resource Center) or RhoC G14V (Missouri S&T Resource Center) using FuGENE (Roche) according to the manufacturer's instructions and then assayed for cell migration using the Cell Migration Colorimetric Assay Kit (Millipore).

Activated Rho Assay.

10 cm Type I collagen coated plates (BD Biosciences) were coated over night (O/N) with 10 μg/ml recombinant hSPARCL1 (R&D) or BSA at 4° C. PC3 cells were then plated on these plates for 6 hours or until equally adherent. Cells were washed twice with ice cold TBS and lysed in 1 ml Rho Buffer (25 mM Hepes, 150 mM NaCl, 1% Igpal, 10 mM MgCl2, 1 mM EDTA, 2% glycerol, PMSF, Sigma protease inhibitor cocktail) for 15 minutes at 4° C. with agitation, passed through a fine gauge needle, cleared by centrifugation, and then incubated with Rhotekin-RBD Protein GST Beads (Cytoskeleton) 0/N at 4° C. with rotation. Both pre-immunoprecipitation and post-immunoprecipitation lysates were collected for analysis Immunoprecipitation lysates were washed three times with ice cold Rho Buffer and then incubated at 70° C. for 10 minutes in 1× NuPage Lysis buffer (Invitrogen) containing PMSF and Sigma protease inhibitor cocktail. PC3 cells were transiently transfected with pcDNA3.1- or hSPARCL1/pcDNA3.1-(Thermo Scientific) using FuGENE (Roche) according to the manufacturer's instructions. Following transfection, cells were incubated with IgG1ₖ (BD Pharmingen) or a blocking antibody to α2β1 (Millipore) for 6 hours on Type 1 collagen plates.

TRAMP Mice and Hi-Myc Mice.

The protocol was approved by the Johns Hopkins University Animal Care and Use Committee. Tissue was obtained from adult euthanized C57BL/6/FVB F1 TRAMP mice (shown), C57BL/6 TRAMP mice and FVB Hi-Myc mice, fixed with 10% neutral buffered formalin, paraffin embedded, and sectioned for IHC.

JHU Prostate Cancer Gleason Grade TMA.

TMAs were constructed from archival tissue from radical prostatectomies performed at Johns Hopkins University between 2000 and 2001. Cases for the TMA were reviewed and selected by a genitourinary pathologist. The largest tumor of the highest grade was selected. In each case, the index tumors of Gleason sum 5, 6, 8 and 9 were spotted in triplicate. Benign adjacent glands were also obtained and spotted in triplicate. 4 μm cut sections were stained for SPARCL1 by IHC as described above. A total of 58 cases were scored by a urologic pathologist for SPARCL1 expression: benign adjacent (n=20), Gleason sum 5 (n=4), Gleason sum 6 (n=16), Gleason sum 8 (n=10), and Gleason sum 9 (n=8). Using an established scoring scheme, SPARCL1 staining intensity was evaluated and assigned an incremental score of 0 (low or absent), 1 (medium), or 2 (strong). Schultz et al., 116 CANCER 5517-26 (2010). Extent of staining was assigned a score for 0-33% (0), 34-66% (1), or 67-100% (2). For each sample, a SPARCL1 score was calculated by adding the intensity score and the extent score (H-score). H-scores were compared using the 1-way ANOVA test with the Bonferroni's post hoc pairwise comparison test. Statistical analyses were performed using GraphPad Software. Statistical tests were two sided and P-values less than 0.05 were considered statistically significant.

JHU Progression TMA: Construction, IHC Staining, and Scoring.

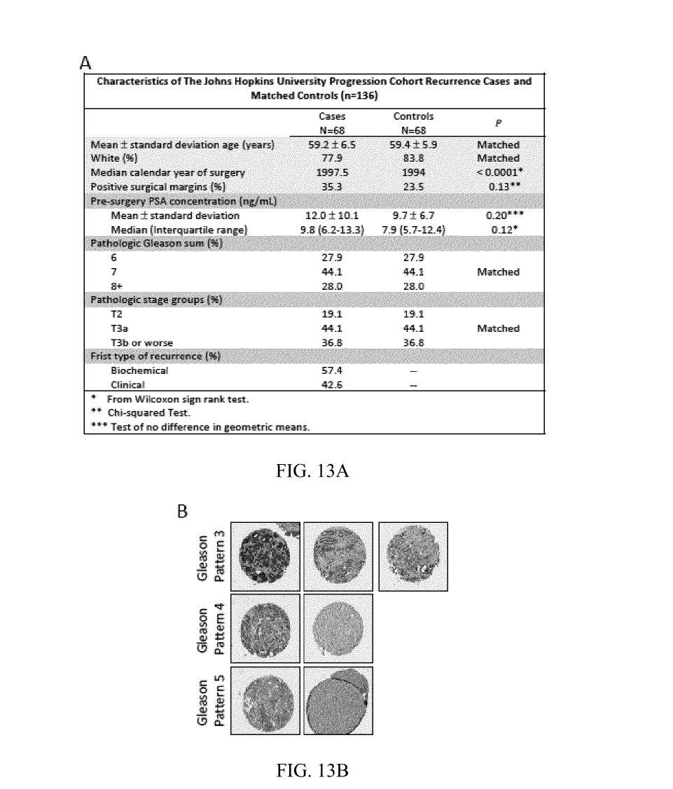

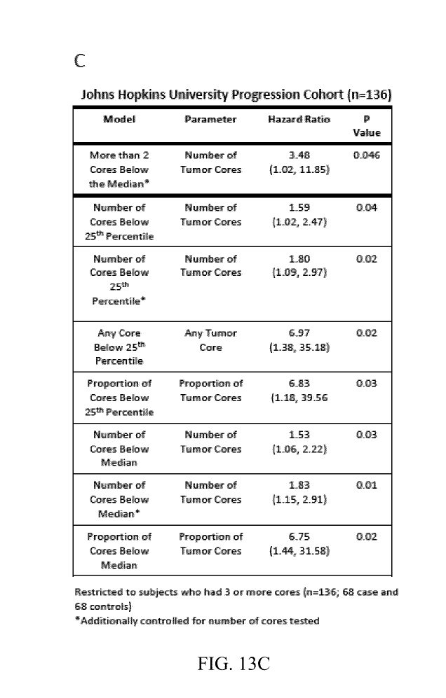

The design of the nested case-control study of prostate cancer recurrence has been described previously. Toubaji et al., 24 MED. PATHOL. 1511-20 (2011). Briefly, included were men who underwent RP for clinically localized prostate cancer at The Johns Hopkins Medical Institutions between 1993 and 2004 and who had not had hormonal or radiation therapy prior to radical prostatectomy or adjuvant therapy prior to recurrence. Cases were men who experienced biochemical recurrence as measured by a re-elevation of serum PSA≧0.2 ng/ml, metastasis, or prostate cancer death after surgery (FIG. 13A). For each case, a control was selected who had not experienced recurrence by the date of the cases' recurrence and who was matched on age, race, pathological stage, and Gleason's sum. Tumors from matched pairs were spotted (0.6 mm) in quadruplicate on the same TMA, which is a strategy that has been shown to provide optimal predictive value for the prostate. Rubin et al., 26 AM. J. SURG. PATHOL. 312-19 (2002). In cases with multifocal tumors, only the index tumor (the dominant tumor with the highest Gleason's sum and usually the largest) was included. Based on a priori sample size calculations, four TMAs were used. One 4 μm section cut from each TMA was stained for SPARCL1 as described above. The extent and intensity of SPARCL1 staining was determined by urologic pathologists using digitized TMAJ software. A single score, called the H score, which integrated both the extent and intensity of SPARCL1 staining, was digitally computed by TMAJ software for each core. After exclusion of technically inadequate TMA cores and men with less than three TMA cores, the final analysis included 68 cases and 68 matched controls. Conditional logistic regression was used to estimate the odds ratio of recurrence taking into account the matching factors and adjusting for year of surgery, pre-surgical PSA level, positive surgical margins, and residual difference in pathologic stage and grade. We categorized each TMA core for each man as being below or at/or above the median H score (calculated among all the cores for all of the controls included on the four TMAs). Analyses were performed using SAS version 9.1 (SAS Institute). Statistical tests were two sided and P-values less than 0.05 were considered to be statistically significant.

Mayo Clinic Progression Analyses: Study Design, Tissue Preparation, RNA Extraction, Microarray Hybridization and Microarray Expression Analysis, and Statistical Analysis.

Study Design.

Patients were selected from a cohort of high-risk RP patients from the Mayo Clinic with a median follow-up of 8.1 years. The cohort was defined as 1010 high-risk men that underwent RP between 2000-2006, of which 73 patients developed metastatic disease as evidenced by positive bone or CT scan. High-risk cohort was defined as preoperative PSA>20 ng/ml, pathological Gleason score 8-10, seminal vesicle invasion (SVI), or GPSM score≧10. Blute et al., 165 J. UROL. 119-25 (2001). The sub-cohort incorporated all 73 metastatic patients and a 20% random sampling of the entire cohort. Of these, tissue specimens were available for 235 patients (n=235). This sub-cohort was previously used to validate a genomic classifier (GC) for predicting metastatic disease at RP.

Tissue Preparation.

Formalin-fixed paraffin embedded (FFPE) samples of human prostate adenocarcinoma prostatectomies were collected from patients at the Mayo Clinic according to an institutional review board-approved protocol. Pathological review of H&E tissue sections was used to guide macrodissection of tumor from surrounding stromal tissue from three to four 10 μm sections. The index lesion was considered the dominant lesion by size.

RNA Extraction and Microarray Hybridization.

For validation cohort, total RNA was extracted and purified using a modified protocol for the commercially available RNeasy FFPE nucleic acid extraction kit (Qiagen). RNA concentrations were calculated using a Nanodrop ND-1000 spectrophotometer (Nanodrop Technologies). Purified total RNA was subjected to whole-transcriptome amplification using the WT-Ovation FFPE system according to the manufacturer's recommendation with minor modifications (NuGen). For the validation only the Ovation® FFPE WTA System was used. Amplified products were fragmented and labeled using the Encore™ Biotin Module (NuGen) and hybridized to Affymetrix Human Exon (HuEx) 1.0 ST GeneChips following manufacturer's recommendations (Affymetrix).

Microarray Expression Analysis.

The normalization and summarization of the microarray samples was done with the frozen Robust Multiarray Average (fRMA) algorithm using custom frozen vectors. These custom vectors were created using the vector creation methods as described previously. Vergara et al., 3 FRONTIERS IN GENETICS 23 (2012). Quantile normalization and robust weighted average methods were used for normalization and summarization, respectively, as implemented in fRMA.

Statistical Analysis.

Given the exon/intron structure of SPARCL1, all probe selection regions (or PSRs) that fall within the genomic span of SPARCL1 were inspected for overlapping this gene. One PSR, 277167, was used for further analysis as a representative PSR for this gene. The PAM (Partition Around Medoids) unsupervised clustering method was used on the expression values of all clinical samples to define two groups of high and low expression of SPARCL1. Statistical analysis on the association of SPARCL1 with clinical outcomes was done using three endpoints (i) Biochemical Recurrence (BCR), defined as two consecutive increases of ≧0.2 ng/ml PSA after RP, (ii) Metastasis (MET), as defined by a positive bone scan and/or CR/MRI evidence of metastatic disease and (iii) Prostate Cancer Specific Mortality (or PCSM). For MET-free survival end point, all patients with metastasis were included in the survival analysis, whereas the controls in the sub-cohort were weighted in a 5-fold manner in order to be representative of patients from the original cohort. For PCSM end point, patients from the cases who did not die by prostate cancer were omitted, and weighting was applied in a similar manner. For BCR, since the case-cohort was designed based on MET-free survival endpoint, re-sampling of BCR patients and sub-cohort was done in order to have a representative of the selected BCR patients from the original cohort.

Other Statistical Analysis.

Statistical analyses were performed using GraphPad Software. Statistical tests were two sided and P-values less than 0.05 were considered statistically significant.

### Results

### Example 1

### Sparcl1 Inhibits Embryonic Epithelial Bud Expansion in the Prostate

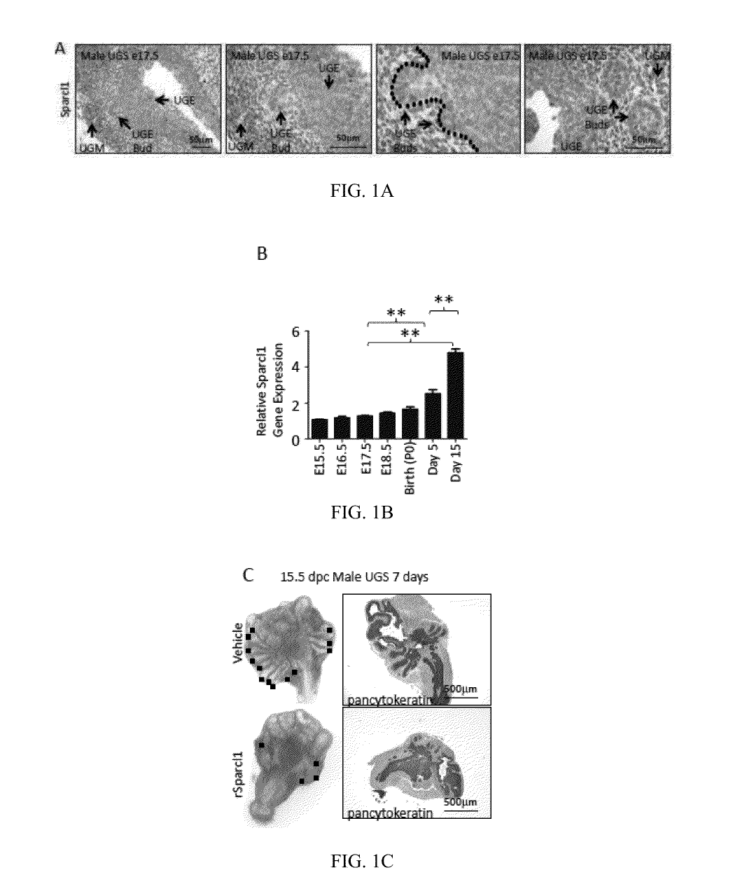

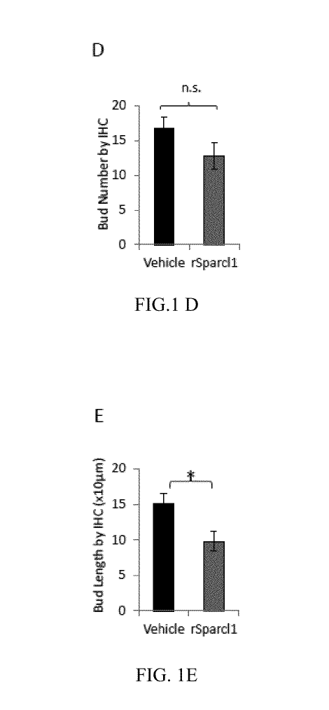

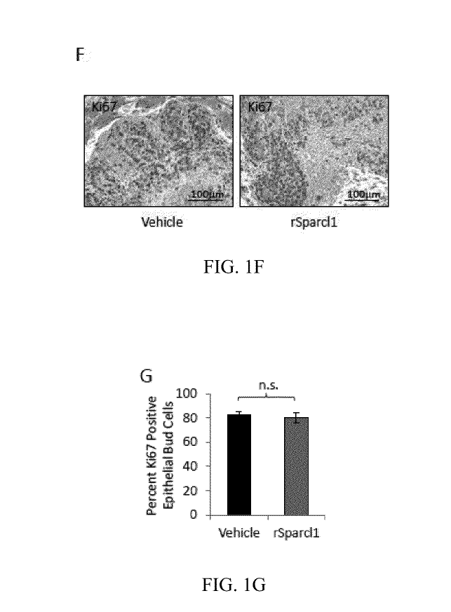

Physiologic prostate growth occurs in an undifferentiated UGS when androgens induce rapid proliferation and invasion of the UGE into the surrounding UGM to form epithelial prostate buds (15, 16). During this phase of development, we previously noted a marked suppression of Sparcl1 gene expression (6). Consistent with this, we observed a discrete loss of Sparcl1 protein expression in the invasive epithelial buds compared to the UGE core (FIG. 1A). Following initial epithelial bud elongation, prostate development continues during branching morphogenesis, a stage that begins in utero and is complete by postnatal day 30 (16). During this phase, we noted a significant rise in Sparcl1 gene expression that inversely correlated with physiologic androgen levels and paralleled the percent completion of branching morphogenesis (FIG. 1B) (17). When added to undifferentiated prostate rudiments (e15.5 male UGS) in organ culture, recombinant Sparcl1 inhibited prostate development. Compared to control treated UGS, Sparcl1 treated UGS exhibited a significant decrease in the number of prostate epithelial buds observed in whole UGS (FIG. 1C). However, when examined by IHC, we noted that Sparcl1 treated UGS had multiple small buds that were not identifiable in whole mount preparations (FIG. 1C, D, F). Consistent with this, bud length was significantly decreased upon exposure to Sparcl1 (FIG. 1E) suggesting that while bud initiation occurs, bud elongation is abrogated. Despite diminished prostate epithelial bud outgrowth, Sparcl1 treated UGS showed epithelial proliferation comparable to control tissue (FIG. 1F, G). Collectively, these observations suggest that the loss of Sparcl1 expression is necessary for epithelial bud migration and elongation into the surrounding mesenchyme during prostate development.

### Example 2

### Sparcl1 Expression is Suppressed during Adult Prostate Regeneration

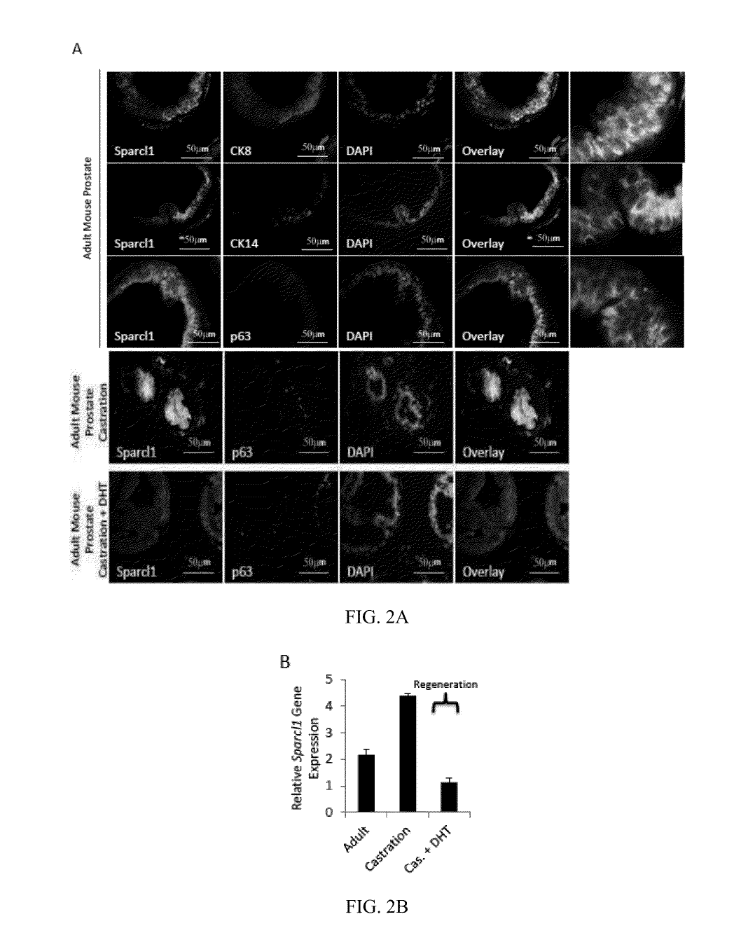

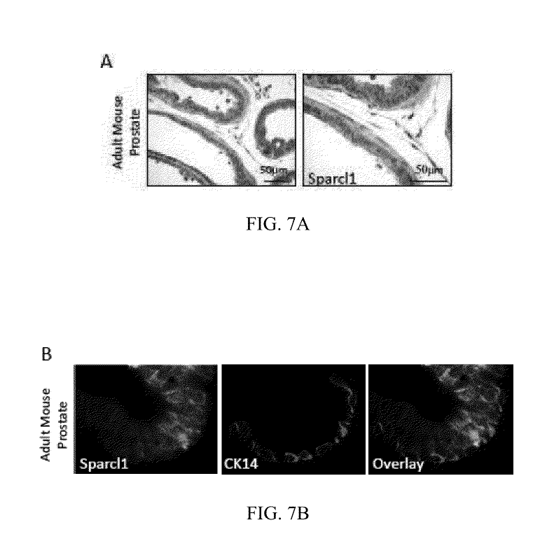

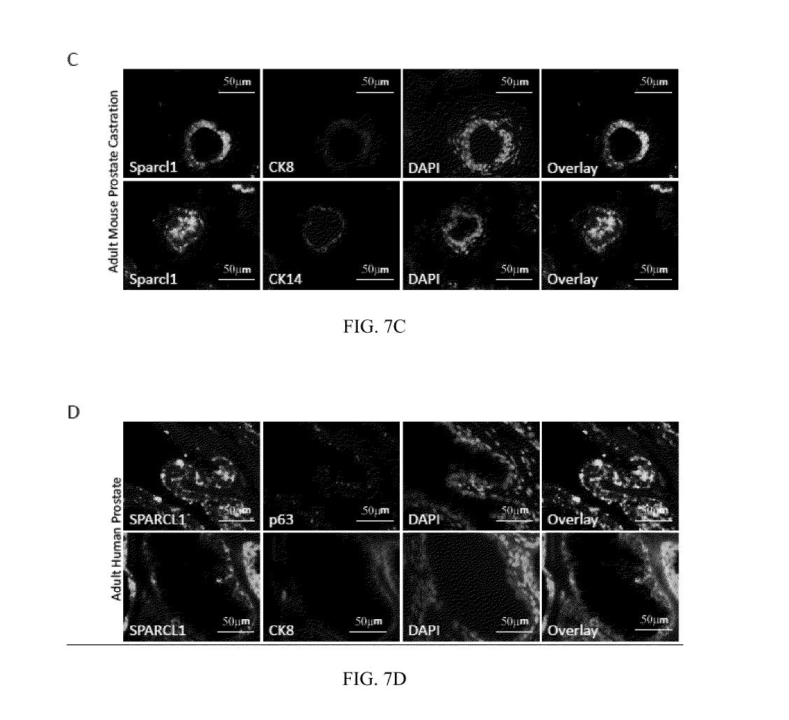

Since Sparcl1 expression is specifically suppressed in migrating epithelial cells during prostate development, we evaluated Sparcl1 expression during androgen mediated regression and regeneration in the adult prostate. In the mature mouse gland, Sparcl1 is expressed predominantly in luminal (CK8 positive) epithelial cells; however, a subpopulation of basal cells (p63 and CK14 positive) co-express Sparcl1 as indicated by IHC and IF (FIG. 2A, 7A,B). SPARCL1 expression in human prostate epithelial cells is similar to that in the mouse (FIG. 7D). Following androgen withdrawal (castration), both Sparcl1 gene and protein expression were elevated (FIG. 2A, B, 7C). Similar to development, Sparcl1 expression was suppressed during androgen-induced prostatic re-growth (FIG. 2A, B). Together these findings indicate that Sparcl1 expression is repressed during phases of androgen stimulated prostatic epithelial growth and invasion in both the embryo and the adult. Considering Sparcl1's role in regulating adhesion and migration, our results suggest that Sparcl1 suppresses epithelial expansion and migration in the prostate.

### Example 3

### SPARCL1 does not Inhibit Proliferation in the Prostate

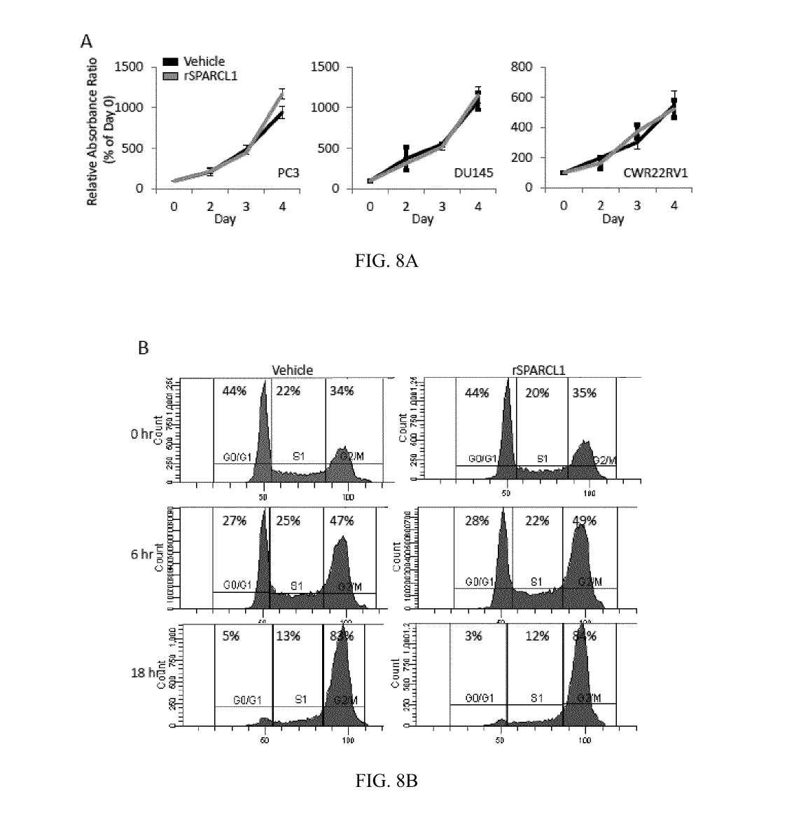

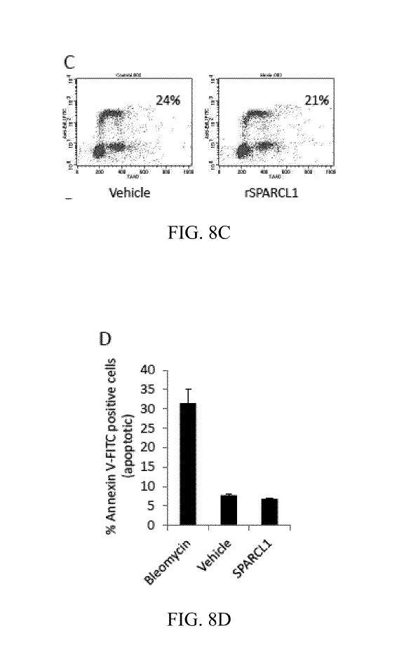

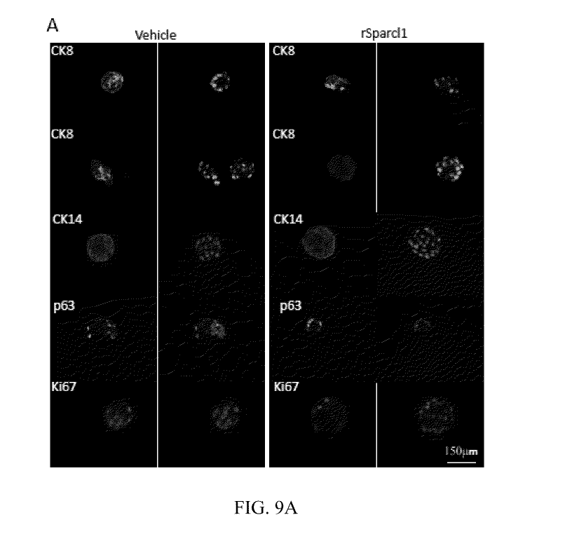

Sparcl1 markedly inhibited prostate epithelial bud elongation; however, comparable expression of proliferation markers in Sparcl1 treated prostate organ cultures suggests that Sparcl1 does not regulate proliferation in the prostate. Since Sparcl1's role in proliferation is varied, we further defined SPARCL1 mediated regulation of prostatic epithelial cell growth (10, 18, 19). We examined cellular proliferation and death in SPARCL1 treated prostate cells and demonstrated that SPARCL1 did not restrict the growth of multiple prostate cancer cell lines (FIG. 8A). SPARCL1 also did not significantly affect cellular proliferation (FIG. 8B) or cell cycle progression (FIG. 8C). SPARCL1 also did not affect cell death (FIG. 8D). Consistent with prostate organ cultures, these data indicate that SPARCL1 does not regulate cellular proliferation or death in the prostate.

### Example 4

### SPARCL1 Inhibits Prostate Cell Adhesion, Migration and Invasion

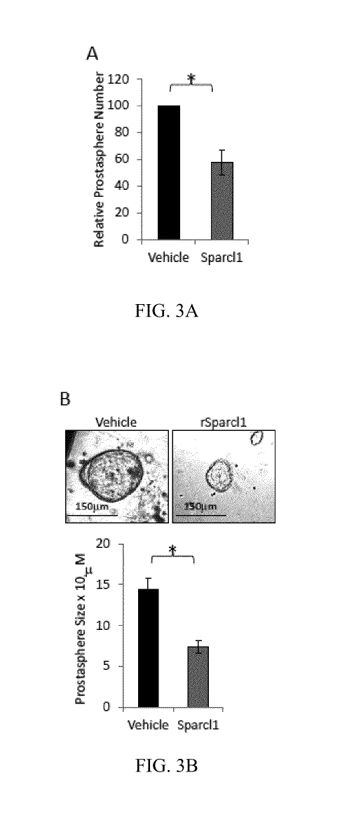

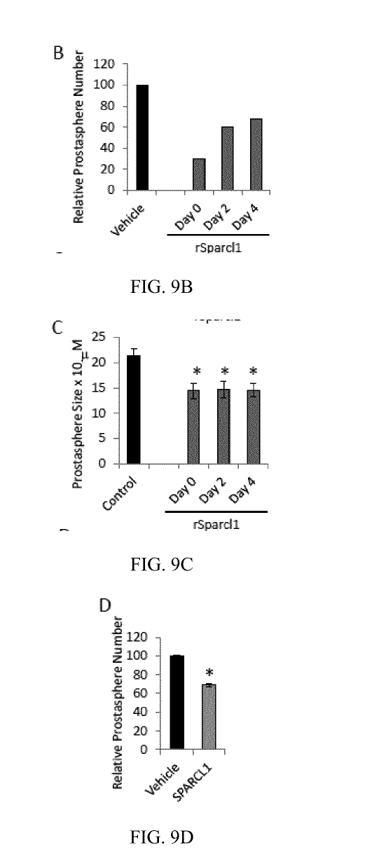

We hypothesized that loss of Sparcl1 expression permits epithelial migration and invasion in prostate organogenesis and regeneration and conversely that Sparcl1 expression restricts these functions in the adult gland. To examine this, we utilized a three-dimensional invasion assay in which single cell epithelial isolates from adult murine prostates can be cultured in Matrigel to form “prostaspheres”. This process is dependent on proliferation and three dimensional migration and invasion into an extracellular matrix. Addition of Sparcl1 to this matrix significantly limited prostasphere number (FIG. 3A) and size (FIG. 3B) without affecting differentiation (CK14, CK8, and p63) or proliferation (Ki67) (FIG. 9A). Augmenting Sparcl1 before or after prostasphere initiation yielded a similar effect (FIG. 9B,C). Similar to the mouse, SPARCL1 restricts prostasphere formation in benign adult human primary prostate epithelial cells (PrEC) (FIG. 9D).

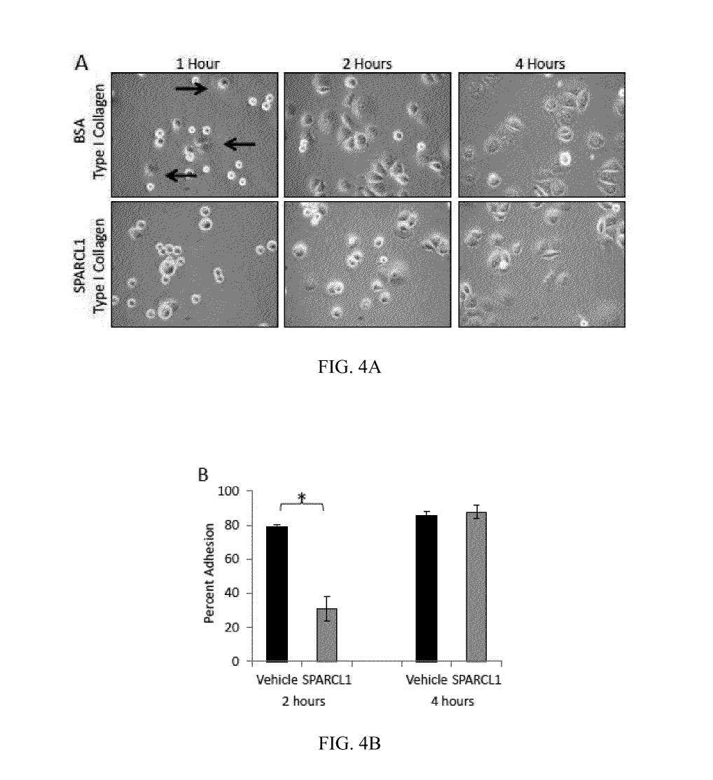

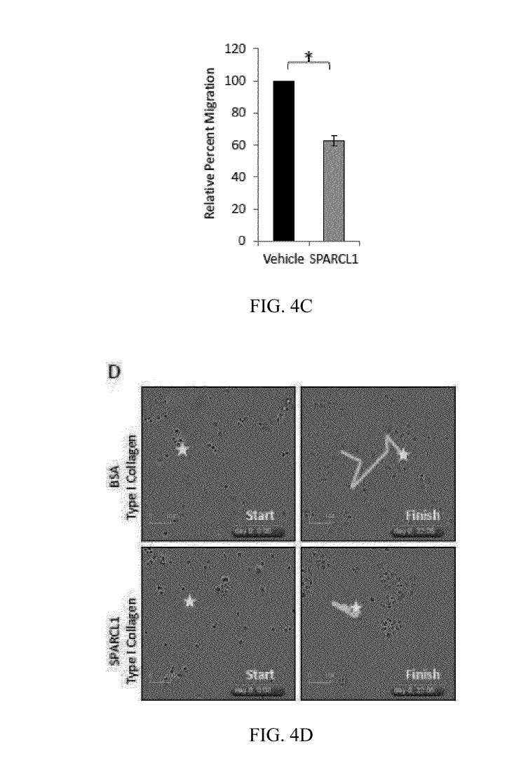

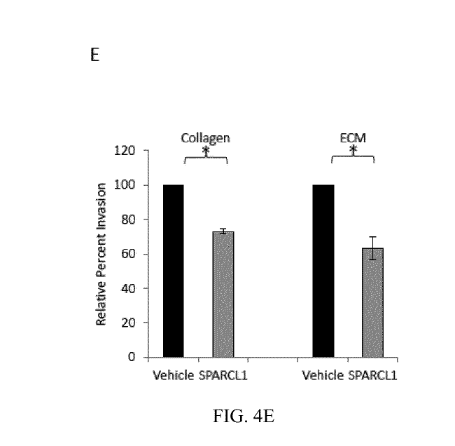

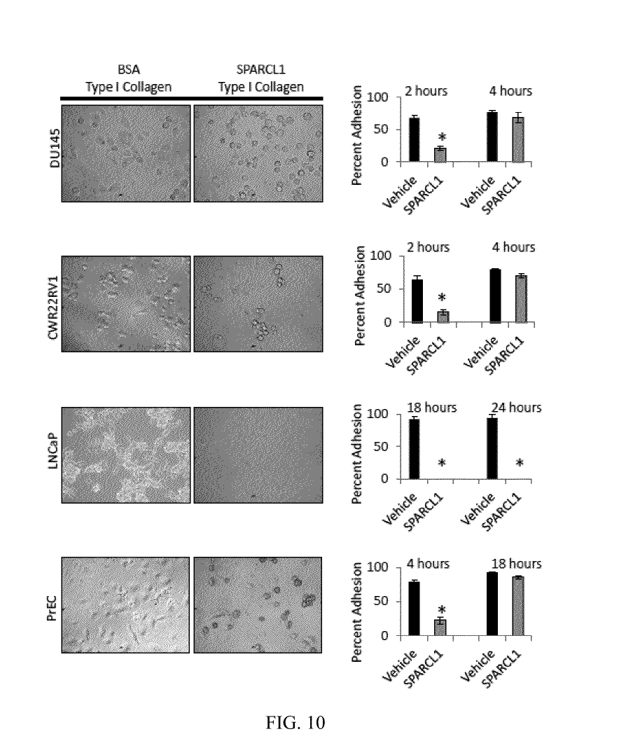

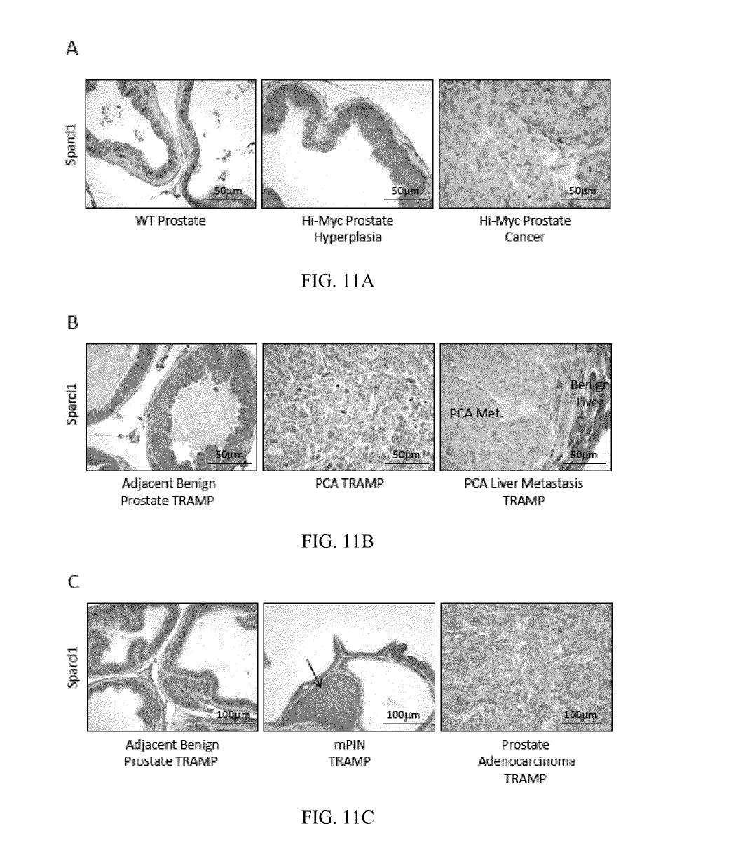

As prostasphere culture requires attachment to an extracellular matrix, and previous studies have shown that Sparcl1 is anti-adhesive (10, 20), we tested the hypothesis that SPARCL1 may prevent prostate cellular adhesion to various extracellular matrices. SPARCL1 delayed or abrogated adhesion of multiple prostate cancer cell lines and primary benign prostate cells to Type I collagen, a key element within the extracellular matrix, and one to which SPARCL1 has been shown to bind (FIG. 4A,B, Videos 1-2, FIG. 10) (11). As adhesion is an initiating event leading to a migratory/invasive phenotype, we further examined how SPARCL1 affects cellular migration and extracellular matrix invasion and found that SPARCL1 inhibited prostate cell migration across a membrane (FIG. 4C). To better elucidate this phenotype, we tested the effects of SPARCL1 on Type I collagen mediated movement. Time-lapse microscopy of prostate cancer cells on a Type I collagen matrix containing SPARCL1 or vehicle demonstrated that SPARCL1 not only delayed adhesion to Type I collagen, but also inhibited migration following adhesion (FIG. 4D, Videos 1-2). Since invasion can be viewed as migration through a matrix, cells that migrate ineffectively should also show defects in invasion. Accordingly, SPARCL1 also inhibited prostate cancer cell invasion as assayed in Type I collagen-based extracellular matrices (FIG. 4E). Collectively, these data support a role for SPARCL1 in regulating the migratory and invasive potential of prostate cancer cells by inhibiting their adhesive and migratory properties.

### Example 5

### SPARCL1 Inhibits RHOC GTPase Mediated Prostate Cancer Cell Migration

RHOC has established roles in promoting cancer cell adhesion, migration, invasion, and metastatic progression (21, 22). Type I collagen engagement of its cognate receptor (α2β1-integrin) has been shown to promote prostate cancer invasion through RHOC (12). As Sparcl1 has been shown to bind to Type I collagen, we hypothesized that SPARCL1 restricts epithelial migration by directly disrupting the function of Type I collagen-RHOC induced migration (11). Following adhesion to a Type I collagen/SPARCL1 matrix, prostate cancer cells exhibited cellular dynamics (Videos 1-2) consistent with inhibition of RHOC but not RHOA (23). To address the possibility that SPARCL1 inhibits Type I collagen induced RHOC activation, we measured RHOC activation in prostate cancer cells following adhesion to one of two different matrices: a Type I collagen matrix containing either BSA (control) or SPARCL1. Specific IP of its active (GTP bound) form demonstrated that RHOC activation was significantly suppressed when cells were grown on a Type I collagen matrix containing SPARCL1 (FIG. 5A). This effect was specific for RHOC as SPARCL1 did not affect activation of RHOA (FIG. 5A). SPARCL1 appeared to suppress migration largely by inhibiting RHOC activation, as the effect of SPARCL1 was rescued by overexpressing a constitutively active RHOC mutant (RHOC G14V) (FIG. 5B). In addition, we found that inhibition of the Type I collagen receptor with a neutralizing antibody resulted in RHOC inhibition that was comparable to that mediated by SPARCL1. In contrast, simultaneous exposure to SPARCL1 and a α2β1-integrin blocking antibody did not further enhance RHOC inhibition suggesting that SPARCL1 and α2β1-integrin function through the same pathway (FIG. 5C). Collectively these data indicate that SPARCL1 inhibits Type I collagen induced RHOC mediated migration.

### Example 6

### SPARCL1 Expression Inversely Correlates with Prostate Cancer Aggressiveness

As SPARCL1 regulated cell invasion, we postulated that SPARCL1 may correlate with and potentially modulate locally aggressive prostate cancers. To examine this, we first evaluated Sparcl1 protein expression in two genetic animal models of prostate cancer. Hi-Myc transgenic mice develop mPIN and locally invasive adenocarcinoma due to prostate specific overexpression of c-Myc (24). In Hi-Myc mice, Sparcl1 expression was decreased in invasive prostate adenocarcinoma (FIG. 11A). We further examined Sparcl1 expression in both primary and metastatic lesions isolated from TRAMP mice; a model with high rates of metastasis (25). Compared to benign adjacent glands and mPIN, Sparcl1 expression was decreased both in invasive prostate adenocarcinoma and in lesions which had metastasized to the liver (FIG. 11B, C). Together, these data indicate Sparcl1 loss predates metastasis and therefore may have prognostic value.

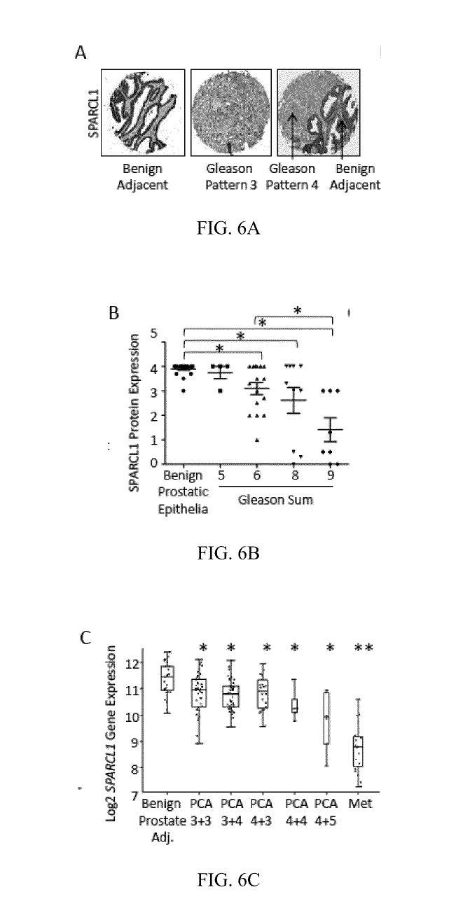

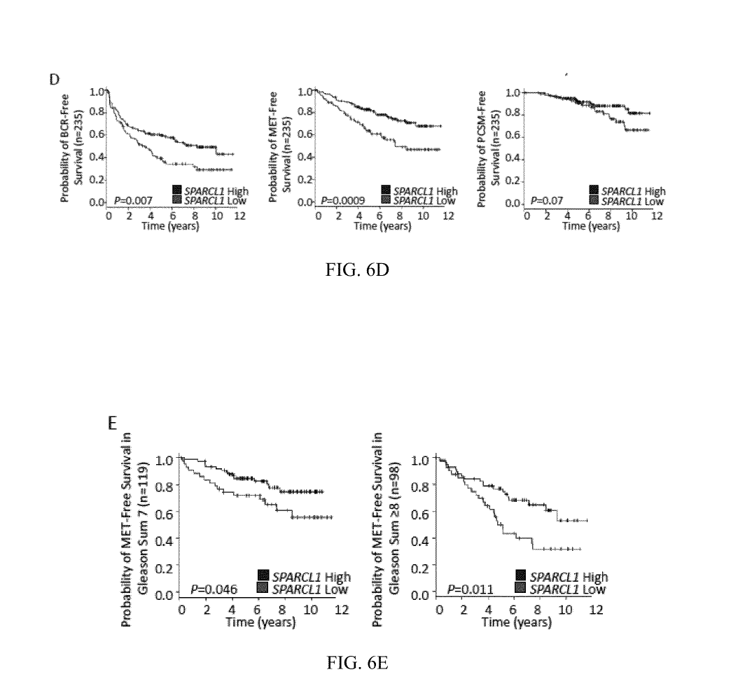

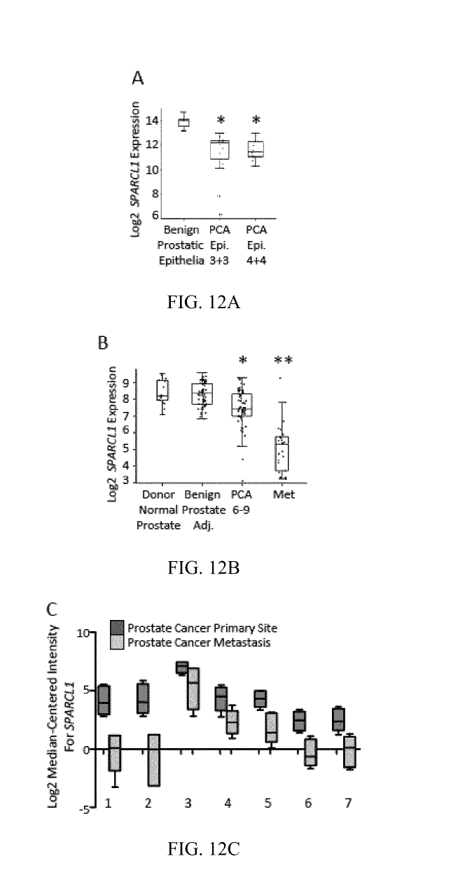

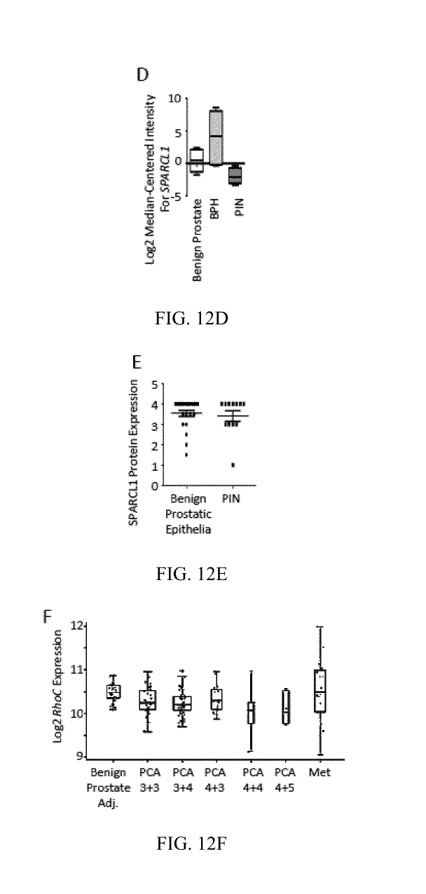

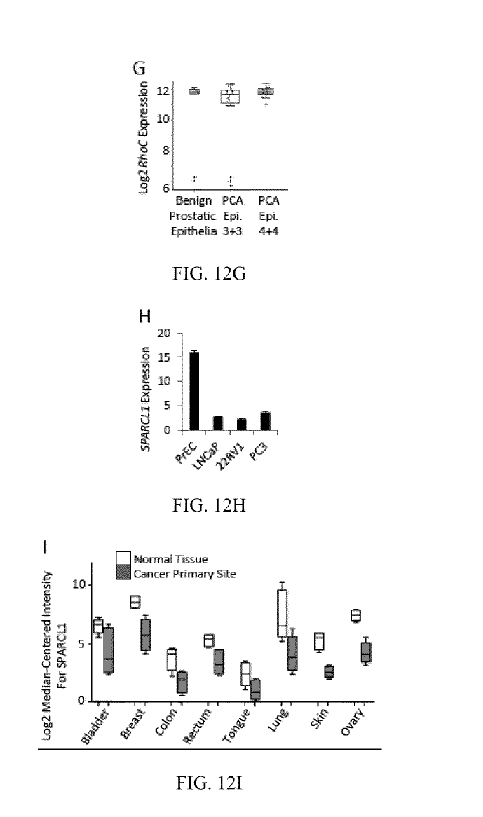

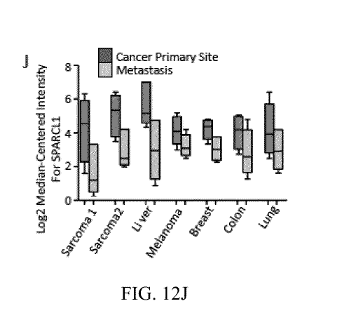

In human prostate cancer, Gleason grade is the strongest single predictor of prostate cancer lethality (1). Low grade (sum 6 or less) rarely progress, whereas men with high grade tumors (sum 8-10) frequently progress to metastasis and death, even after radical treatment (1, 26). IHC analysis of SPARCL1 expression on TMAs demonstrated a statistically significant inverse correlation between Gleason grade and SPARCL1 expression (FIG. 6A,B). Consistent with this, analyses of 10 datasets indicated that parallel to protein expression, SPARCL1 gene expression declined continuously as grade increased with the most striking loss seen in metastatic lesions (FIG. 6C, 12A-C) (27-36; Oncomine™) In contrast, SPARCL1 gene expression was not significantly lost in BPH or PIN (FIG. 12D, E) (27, 28, 37; Oncomine™). Interestingly, gene profiling data from the same cohorts showed that RHOC gene expression did not correlate with prostate cancer grade (FIG. 12F,G) (27, 28), suggesting that alterations in RHOC activity as opposed to expression levels, mediate RHOC induced metastatic progression.

We additionally investigated SPARCL1 expression in other primary cancers and their metastases and also found it decreased in a variety of cancer types, including bladder (39), breast (40), and lung (41; Oncomine™) (FIG. 12I) with further suppression in metastases compared to primary tumors (FIG. 12J) (41-47; Oncomine™). These observations suggest that SPARCL1 suppression is a conserved and critical step in cancer progression to metastasis.

### Example 7

### Loss of SPARCL1 Expression is an Independent Marker of Recurrence after Prostatectomy

A subset of men with clinically localized prostate cancer experience disease recurrence even after primary treatment. Although current models incorporating Gleason grade, pathologic stage and other clinical parameters predict recurrence (48), further delineation of risk is needed. We postulated that SPARCL1 loss could add prognostic power to these traditionally used variables. We examined SPARCL1 expression by IHC in a nested case-control matched cohort designed to evaluate prognostic risk factors for recurrence following prostatectomy (defined as PSA≧0.2 ng/ml, metastasis, or prostate cancer death) independent of Gleason grade, pathologic stage, age and other clinical variables (JHU progression array) (32) (FIG. 13A). We found that loss of SPARCL1 expression in prostate adenocarcinoma was independently associated with a 3.48 fold (95% CI 1.02-11.85; P=0.046) higher risk of prostate cancer recurrence (FIG. 13A-C).

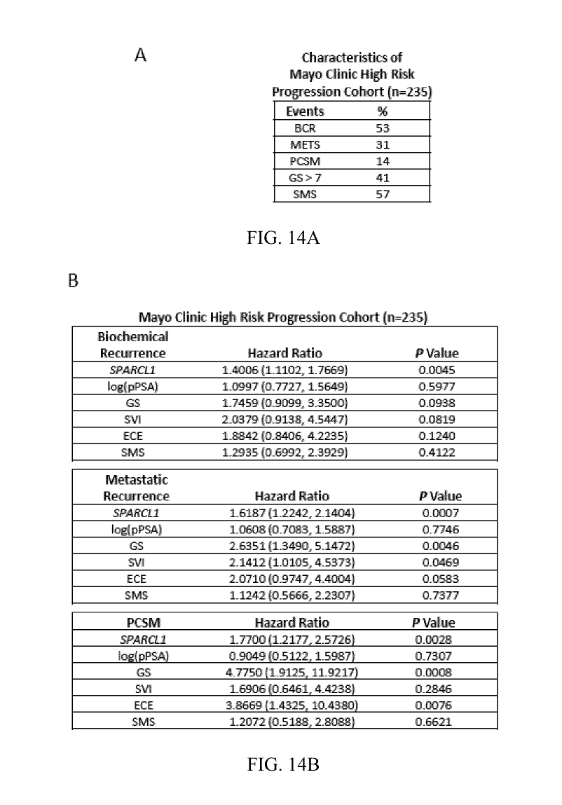

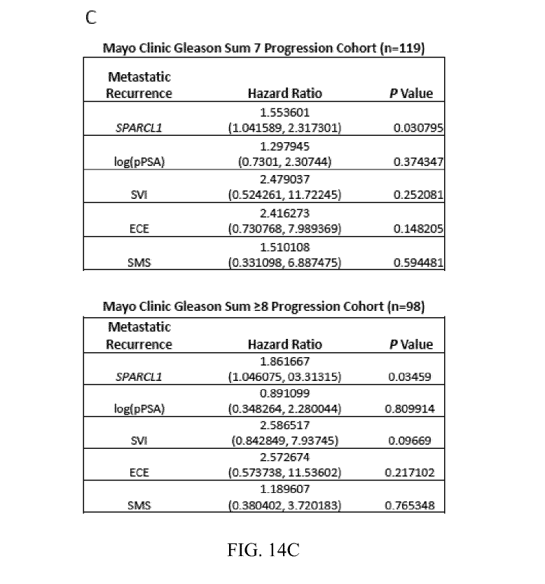

We validated this finding in an independent cohort utilizing Affymetrix exon microarray analysis in a prospectively-designed study of high-risk men who underwent radical prostatectomy (RP) at the Mayo Clinic (FIG. 14A) (58). We evaluated the prognostic utility of SPARCL1 using three clinical endpoints: biochemical recurrence (BCR), metastatic disease (MET) as defined by a positive bone scan and/or CR/MRI evidence of metastatic disease, and prostate cancer-specific mortality (PCSM). Kaplan-Meier analyses show loss of SPARCL1 is a powerful single-gene predictor of aggressive prostate cancer (FIG. 6D). For BCR, loss of SPARCL1 expression was associated with a median time-to-progression of 3.5 yrs compared to greater than 8 yrs for high SPARCL1 expressing men. Similarly, for MET-free survival, men with loss of SPARCL1 expression had 5 yr MET-free survival of ˜60% vs. ˜80% for men with high SPARCL1 expression. These data suggest that even in a high risk cohort where most individuals are expected to experience recurrence at some point after surgery, loss of SPARCL1 expression defines a subgroup where BCR will occur sooner and the risk for developing metastatic disease and prostate cancer death is significantly higher.

Furthermore, multivariable Cox regression analyses of the Mayo Clinic cohort confirmed the JHU observation that loss of SPARCL1 expression is independently prognostic of prostate cancer aggressiveness with significant hazard ratios for predicting BCR, MET and PCSM (HR 1.40, P=0.0045; HR 1.62, P=0.0007; HR 1.77, P=0.0028, respectively) (FIG. 14B). In groups of men stratified by Gleason score (sum 7 and sum≧8), SPARCL1 suppression significantly identified men at increased risk of developing metastatic disease (Gleason sum 7 HR 1.55, P=0.03 and Gleason sum≧8 HR 1.86 P=0.03) (FIG. 6E). In fact, in these Gleason subgroups, multivariable Cox regression analyses of SPARCL1 and standard prognostic factors including stage demonstrated that loss of SPARCL1 expression was the only statistically significant predictor of recurrence (FIG. 14C).

### REFERENCES

1. Eggener S E, et al. (2011) Predicting 15-year prostate cancer specific mortality after radical prostatectomy. J Urol 185:869-875.
2. Loeb S, et al. (2010) What are the outcomes of radical prostatectomy for high-risk prostate cancer? Urology 76:710-714.
3. Pierorazio P M, et al. (2010) Long-term survival after radical prostatectomy for men with high Gleason sum in pathologic specimen. Urology 76:715-721.
4. D'Amico A V, et al. (1998) Analyzing outcome-based staging for clinically localized adenocarcinoma of the prostate. Cancer 83:2172-2180.
5. Pierorazio P M, et al. (2012) Preoperative characteristics of high-Gleason disease predictive of favourable pathological and clinical outcomes at radical prostatectomy. BJU Int epub.
6. Schaeffer E M, et al. (2008) Androgen-induced programs for prostate epithelial growth and invasion arise in embryogenesis and are reactivated in cancer. Oncogene 27:7180-7191.
7. Pritchard C, et al. (2009) Conserved gene expression programs integrate mammalian prostate development and tumorigenesis. Cancer Res 69:1739-1747.
8. Cunha G R (2008) Mesenchymal-epithelial interactions: past, present, and future. Differentiation 76:578-586.
9. Nelson P S, et al. (1998) Hevin, an antiadhesive extracellular matrix protein, is down-regulated in metastatic prostate adenocarcinoma. Cancer Res 58:232-236.
10. Sullivan M M, Puolakkainen P A, Barker T H, Funk S E, & Sage E H (2008) Altered tissue repair in hevin-null mice: inhibition of fibroblast migration by a matricellular SPARC homolog. Wound Repair Regen 16:310-319.
11. Hambrock H O, et al. (2003) SC1/hevin. An extracellular calcium-modulated protein that binds collagen I. J Biol Chem 278:11351-11358.
12. Hall C L, et al. (2008) Type I collagen receptor (alpha2beta1) signaling promotes prostate cancer invasion through RhoC GTPase. Neoplasia 10:797-803.
13. Armstrong T, et al. (2004) Type I collagen promotes the malignant phenotype of pancreatic ductal adenocarcinoma. Clin Cancer Res 10:7427-7437.
14. Said N, et al. (2009) The role of SPARC in the TRAMP model of prostate carcinogenesis and progression. Oncogene 28:3487-3498.
15. Meeks J J & Schaeffer E M (2011) Genetic regulation of prostate development. J Androl 32:210-217. 14
16. Prins G S & Putz O (2008) Molecular signaling pathways that regulate prostate gland development. Differentiation 76:641-659.
17. Pritchard C C & Nelson P S (2008) Gene expression profiling in the developing prostate. Differentiation 76:624-640.
18. Claeskens A, et al. (2000) Hevin is down-regulated in many cancers and is a negative regulator of cell growth and proliferation. Br J Cancer 82:1123-1130.
19. Esposito I, et al. (2007) Tumor-suppressor function of SPARC-like protein 1/Hevin in pancreatic cancer. Neoplasia 9:8-17.
20. Girard J P & Springer T A (1996) Modulation of endothelial cell adhesion by hevin, an acidic protein associated with high endothelial venules. J Biol Chem 271:4511-4517.
21. Wu M, Wu Z F, Rosenthal D T, Rhee E M, & Merajver S D (2010) Characterization of the roles of RHOC and RHOA GTPases in invasion, motility, and matrix adhesion in inflammatory and aggressive breast cancers. Cancer 116:2768-2782.
22. Hakem A, et al. (2005) RhoC is dispensable for embryogenesis and tumor initiation but essential for metastasis. Genes Dev 19:1974-1979.
23. Vega F M, Fruhwirth G, Ng T, & Ridley A J (2011) RhoA and RhoC have distinct roles in migration and invasion by acting through different targets. J Cell Biol 193:655-665
24. Ellwood-Yen K, et al. (2003) Myc-driven murine prostate cancer shares molecular features with human prostate tumors. Cancer Cell 4:223-238.
25. Gingrich J R, et al. (1996) Metastatic prostate cancer in a transgenic mouse. Cancer Res 56:4096-4102.
26. Pound C R, et al. (1999) Natural history of progression after PSA elevation following radical prostatectomy. JAMA 281:1591-1597.
27. Taylor B S, et al. (2010) Integrative genomic profiling of human prostate cancer. Cancer Cell 18:11-22.
28. Ross A E, et al. (2011) Gene expression pathways of high grade localized prostate cancer. Prostate 71:1568-1577).
29. Chandran U R, et al. (2005) Differences in gene expression in prostate cancer, normal appearing prostate tissue adjacent to cancer and prostate tissue from cancer free organ donors. BMC Cancer 5:45.
30. Yu Y P, et al. (2004) Gene expression alterations in prostate cancer predicting tumor aggression and preceding development of malignancy. J Clin Oncol 22:2790-2799.
31. Holzbeierlein J, et al. (2004) Gene expression analysis of human prostate carcinoma during hormonal therapy identifies androgen-responsive genes and mechanisms of therapy resistance. Am J Pathol 164:217-227.
32. LaTulippe E, et al. (2002) Comprehensive gene expression analysis of prostate cancer reveals distinct transcriptional programs associated with metastatic disease. Cancer Res 62:4499-4506.
33. Lapointe J, et al. (2004) Gene expression profiling identifies clinically relevant subtypes of prostate cancer. Proc Natl Acad Sci USA 101:811-816.
34. Varambally S, et al. (2005) Integrative genomic and proteomic analysis of prostate cancer reveals signatures of metastatic progression. Cancer Cell 8:393-406.
35. Ramaswamy S, et al. (2001) Multiclass cancer diagnosis using tumor gene expression signatures. Proc Natl Acad Sci USA 98:15149-15154.
36. Ramaswamy S, Ross K N, Lander E S, & Golub T R (2003) A molecular signature of metastasis in primary solid tumors. Nat Genet 33:49-54.
37. Tomlins S A, et al. (2007) Integrative molecular concept modeling of prostate cancer progression. Nat Genet 39:41-51.
38. Karlsson R, Pedersen E D, Wang Z, & Brakebusch C (2009) Rho GTPase function in tumorigenesis. Biochim Biophys Acta 1796:91-98.
39. Sanchez-Carbayo M, Socci N D, Lozano J, Saint F, & Cordon-Cardo C (2006) Defining molecular profiles of poor outcome in patients with invasive bladder cancer using oligonucleotide microarrays. J Clin Oncol 24:778-789.
40. Richardson A L, et al. (2006) X chromosomal abnormalities in basal-like human breast cancer. Cancer Cell 9:121-132.
41. Bhattacharjee A, et al. (2001) Classification of human lung carcinomas by mRNA expression profiling reveals distinct adenocarcinoma subclasses. Proc Natl Acad Sci USA 98:13790-13795.
42. Radvanyi L, et al. (2005) The gene associated with trichorhinophalangeal syndrome in humans is overexpressed in breast cancer. Proc Natl Acad Sci USA 102:11005-11010.
43. Segal N H, et al. (2003) Classification and subtype prediction of adult soft tissue sarcoma by functional genomics. Am J Pathol 163:691-700.
44. Segal N H, et al. (2003) Classification of clear-cell sarcoma as a subtype of melanoma by genomic profiling. J Clin Oncol 21:1775-1781.
45. Liao Y L, et al. (2008) Identification of SOX4 target genes using phylogenetic footprinting-based prediction from expression microarrays suggests that overexpression of SOX4 potentiates metastasis in hepatocellular carcinoma. Oncogene 27:5578-5589.
46. Xu L, et al. (2008) Gene expression changes in an animal melanoma model correlate with aggressiveness of human melanoma metastases. Mol Cancer Res 6:760-769.
47. Ki D H, et al. (2007) Whole genome analysis for liver metastasis gene signatures in colorectal cancer. Int J Cancer 121:2005-2012.
48. Han M, et al. (2003) Biochemical (prostate specific antigen) recurrence probability following radical prostatectomy for clinically localized prostate cancer. J Urol 169:517-523.
49. Ben-Porath I, et al. (2008) An embryonic stem cell-like gene expression signature in poorly differentiated aggressive human tumors. Nat Genet 40:499-507.
50. Iiizumi M, et al. (2008) RhoC promotes metastasis via activation of the Pyk2 pathway in prostate cancer. Cancer Res 68:7613-7620.
51. van Golen K L, et al. (1999) A novel putative low-affinity insulin-like growth factor-binding protein, LIBC (lost in inflammatory breast cancer), and RhoC GTPase correlate with the inflammatory breast cancer phenotype. Clin Cancer Res 5:2511-2519.
52. Kamai T, et al. (2003) Significant association of Rho/ROCK pathway with invasion and metastasis of bladder cancer. Clin Cancer Res 9:2632-2641.
53. Shikada Y, et al. (2003) Higher expression of RhoC is related to invasiveness in non-small cell lung carcinoma. Clin Cancer Res 9:5282-5286.

We claim:

1. A method for determining a likelihood of prostate cancer recurrence in a patient following prostectomy comprising the steps of:
a. obtaining a biological sample from the patient;
b. subjecting the sample to an assay for detecting SPARCL1 expression; and
c. determining that prostate cancer is likely to recur if SPARCL1 expression is decreased relative to a reference non-prostate cancer sample.

2. A method for predicting metastasis in prostate cancer patient comprising the steps of:
a. obtaining a biological sample from the patient;
b. subjecting the sample to an assay for detecting SPARCL1 expression; and
c. determining that metastasis is likely to occur if SPARCL1 expression is decreased relative to a reference non-metastatic prostate cancer sample.

3. A method for identifying prostate cancer lesions with metastatic potential in a patient comprising the steps of:
a. obtaining a biological sample from the patient;
b. subjecting the sample to an assay for detecting SPARCL1 expression; and
c. determining that the prostate cancer lesions have metastatic potential if SPARCL1 expression is decreased relative to a reference non-metastatic prostate cancer sample.

4. A method for diagnosing prostate cancer or a likelihood thereof in a patient comprising the steps of:
a. obtaining a biological sample from the patient;
b. subjecting the sample to an assay for detecting SPARCL1 expression; and
c. determining that the cancer lesions have metastatic potential if SPARCL1 expression is decreased relative to a reference non-prostate cancer sample.

5. A method for determining a likelihood of prostate cancer recurrence in a patient following prostectomy comprising the steps of:
a. obtaining a prostate tissue sample from the patient;
b. performing an assay on the sample to measure SPARCL1 expression;
c. providing a reference non-prostate cancer tissue sample;
d. comparing the level of SPARCL1 expression from the prostate tissue sample of the patient to the level of SPARCL1 expression in the reference non-prostate cancer tissue sample; and
e. determining that prostate cancer is likely to recur when the level of SPARCL1 expression in the prostate tissue sample of the patient is decreased relative to the level of SPARCL1 expression in the reference non-prostate cancer tissue sample.

6. A method for predicting metastasis in prostate cancer patient comprising the steps of:
a. obtaining a prostate tissue sample from the patient;
b. performing an assay on the sample to measure SPARCL1 expression;
c. providing a reference non-prostate cancer tissue sample;
d. comparing the level of SPARCL1 expression from the prostate tissue sample of the patient to the level of SPARCL1 expression in the reference non-prostate cancer tissue sample; and
e. determining that metastasis is likely to occur when the level of SPARCL1 expression in the prostate tissue sample of the patient is decreased relative to the level of SPARCL1 expression in the reference non-prostate cancer tissue sample.

7. A method for identifying cancer lesions with metastatic potential in a patient comprising the steps of:
a. obtaining a prostate tissue sample from the patient;
b. performing an assay on the sample to measure SPARCL1 expression;
c. providing a reference non-prostate cancer tissue sample;
d. comparing the level of SPARCL1 expression from the prostate tissue sample of the patient to the level of SPARCL1 expression in the reference non-prostate cancer tissue sample; and
e. determining that the cancer lesions have metastatic potential when the level of SPARCL1 expression in the prostate tissue sample of the patient is decreased relative to the level of SPARCL1 expression in the reference non-prostate cancer tissue sample.

8. A method for identifying a patient as having prostate cancer comprising the steps of:
a. obtaining a prostate tissue sample from the patient;
b. performing an assay on the sample to measure SPARCL1 expression;
c. providing a reference non-prostate cancer tissue sample;
d. comparing the level of SPARCL1 expression from the prostate tissue sample of the patient to the level of SPARCL1 expression in the reference non-prostate cancer tissue sample; and
e. identifying the patient as having prostate cancer when the level of SPARCL1 expression in the prostate tissue sample of the patient is decreased relative to the level of SPARCL1 expression in the reference non-prostate cancer tissue sample.

9. The method of claim 1, wherein the reference non-prostate cancer tissue sample is a sample from benign prostate tissue.

10. The method of claim 9, wherein the benign prostate tissue is from the patient.


SEQUENCE LISTING (4 sequences)

SEQ ID NO: 1 - DNA, 22 bp, Artificial Sequence
  Forward primer for human SPARCL1
gttccttcacagattctaacca

SEQ ID NO: 2 - DNA, 21 bp, Artificial Sequence
  Reverse primer to human SPARCL1
tttactgctcctgttcaactg

SEQ ID NO: 3 - DNA, 20 bp, Artificial Sequence
  Forward primer to human SPARCL1
atcattccaaaccaactgct

SEQ ID NO: 4 - DNA, 20 bp, Artificial Sequence
  Reverse primer to human SPARCL1
gactgttcatggctttcctc